SEQUENCE LISTING (38 sequences)

SEQ ID NO: 1 - DNA, 276 bp, Oryza sativa
atgggtttgcaggaggagtttgaggagttcgccgagaaggccaagaccttgcctgacacaatttccaacgaggacaagctgcttctctatggcctctacaagcaggcaaccgttggcccggttaccactgggcgcccaggtattttcaacctgaaagacagatacaaatgggatgcttggaaggccgttgaagggaaatccaaggaggaagctatggccgattacatcaccaaggtgaagcagctgctggaggaggcttctgcatccacttcttag

SEQ ID NO: 2 - protein, 91 aa, Oryza sativa
MGLQEEFEEFAEKAKTLPDTISNEDKLLLYGLYKQATVGPVTTGRPGIFNLKDRYKWDAWKAVEGKSKEEAMADYITKVKQLLEEASASTS

SEQ ID NO: 3 - DNA, 1665 bp, Oryza sativa
ggcgtccattgatcttcctcccccctccaactccaagaaatcgactgacgccaacgagaacgagacgccatcgctgccatgggtttgcaggtaaattaaacgaatcgtcttcctgcttcttcttcttcttttcccttctcgtgattttctttgtgttttcttgagcggatgcgcataggttgaatggatattactcctccgcttgttgaaatcaatctacctgcagatttgctggttgtttatcgtttaggattttcttccctttggcatgcaatatatatcttgtttggagtaccatactaccatgtggtttaacagtatgttaatttgtaattaaccaatctaatctacctgctgttcgttcctctgttggttaatttagtagtgacagtgttctggatcaagtagctaggtaggtgcagtagtgataaaagatcaaacctttttgtgctttagactttagaggagccatcagaaactgcaattgatgcctttgttatcctgttcttctgaactccaaatatgcagagcagtttcctgaggtaaacaaattcaagtcgattctgaacaaattagttgttgttcttgttgtgcaggaggagtttgaggagttcgccgagaaggccaagaccttgcctgacacaatttccaacgaggacaagctgcttctctatggcctctacaagcaggcaaccgttggcccggttaccactggtatatataggccttctgctcttcttgaaacgatgcattcatcaattgagctaattaatttgctgctgttcaagtgctaagattccatatgctttcatgatgatcaggcaagattagctttcatatagttgcatctgaatcactgttttgtgttttgactaaaaaggattatcgatctgcttaagttttgccacaaatttagtgcttgatgagtgtaaactcatctatccttgtaatctgcttgtcaatttggggtgtttaatattacaatttccagaggaattattcacctattacagtactaagtggtggttgtcatttgaaattgtagtaacatatggtattcttatgtttctccccgatggggactttgcttagcagctgttctactttttttttaaatcatgacctttcaagttccaaaggcaatgcttttggacttgtttcgtaatacagacaccaaagatcacattttatcctcgagttatcgtatctaacaattcagtttcatctttcagggcgcccaggtattttcaacctgaaagacagatacaaatgggatgcttggaaggccgttgaaggtatatgccgatctctttaagtcctcaaaaacatatatcagctaaactatataactttgtcatatctgcaaactcccatcaatgcttacaaatccaaatcatggtgaattcagggaaatccaaggaggaagctatggccgattacatcaccaaggtgaagcagctgctggaggaggcttctgcatccacttcttaggctattatgaacaacaccatcaatagtgctgcatatatgtaccaataaacataaatactataccatcatcaggcgctttgtgatcatcccttctctgtacttgtaatagtttcaagcagactcggtcctgttaattgatcatgactgttattaagtttgtccgtcttttctaaa

SEQ ID NO: 4 - DNA, 270 bp, Zea mays
atgggtctgcaggaggaatttgaggagcatgctgagaaggcaaagaccttgcctgagagcacgagcaatgagaacaagctgatcctctacgggctctacaagcaagccacggttggagatgtgaataccgatcgtccgggcatattctaccaaaaggacagggccaagtgggatgcctggaaggctgttgaaggcaaatcgaaggatgaggcgatgaatgactacatcaccaaggtgaagcagctccaggaggaggctgccgcctcttaa

SEQ ID NO: 5 - protein, 89 aa, Zea mays
MGLQEEFEEHAEKAKTLPESTSNENKLILYGLYKQATVGDVNTDRPGIFYQKDRAKWDAWKAVEGKSKDEAMNDYITKVKQLQEEAAAS

SEQ ID NO: 6 - DNA, 2670 bp, Zea mays
cataatgactccagtatcgctctatataacaagtggatccgatctagtcgccagtcgtcgtcgttaaaaccgagaagagaccaggccgaaggaaaacaatcctgaaaccaagccatgggtctgcaggtacacggctcatcactctctaaatcgccatattctgctactcgcttgttgaagatcgaacgtttattcatctgatggtcaccaagactggttctttgtttccatttggttctgaatctccagttccgtttcgattcttgcactgtaccccaacccacagtagtgtctatacgaggttcgtttttaggttctccagagaaacgatctagacttcaaatccgctgctgcgtggtcttattttccatctagatttgtatgcgtagccaagatctggtggattcagactgttggctctttgtaccctttatgttcatgcttaagaggggatccaatgcttttgcgccgccctaactctcgagcggttcatcatgcctttggatgtaagatggaaaattccccgtttgttttaagtattttgcgacttatcggttttccatataattctatagagaaatagagtggcgaagcgcgtgggtcaaagtctggattgcttaaaatggtttgcatgaaagatttaccttgtgaaatagtttagcatagtattgcaatcgtggtcccctaggaatatgagctgtatctctgttgcaggaggaatttgaggagcatgctgagaaggcaaagaccttgcctgagagcacgagcaatgagaacaagctgatcctctacgggctctacaagcaagccacggttggagatgtgaataccggttagtatgtaatcacgcagctaattttgttctcttcttttcagttccctttctaagtttggtgtgtggatgaagctcttggtttgcagaaacctaattcaatttaagtgtcatggctcaaaactattcctagcatggatagtgtttaataactttatgtgtcataatacgaattccctgaaaggttgacgatttgcttcctctgtataaattaaacagcagggcctgctagtttgcttttgaagtttaacgatttaatttagaaatctgaacagtggtgaatatgtattgtgaatccgtgataaaatataaaatagggggaaatcctaagaatataaaatgcgggcaaaatcctagtaagcaaacaagacggtattataaagaataagatgaacatatttactgtaatatctttttatttcttcccaagtttggacaacaaattttgaccatgattctagggcttgtttatgtagtacatagcaacagaagttcagcagtaatggaacagggatgtattagatgttgaccaacctcttggggttcaactaatactgaattgtagatttctatgctctgtcctgcaacatctatttactgatgaaatttgctgcaccgagatgcagttgaatggtaaattcaggttcatagggtttggctgagtaacttaacatagagttaaaaaaatgtaatgttcatatcattatatagttcaaaacactcaaatgtgttttttcttccatatgtcagttgtaaccaagtcatgattatgctctgtttgcgttgtactaaatgaacctctagtctgttcgaccagttgccattaactcatgggaccatgatagtgtctgttgaaaaacttatctgcgcatcttttctgtttacaatagtatatattttttaacacagctgtgcttacatgtttgttcgttaataaacttttgaagatcgtccgggcatattctaccaaaaggacagggccaagtgggatgcctggaaggctgttgaaggttcatacactcatctgatcgttgtattttgggctgtttaaattttttttaccacaatactagtgttctacatggatgtatctattccaatagcatcacaaggtattacagtacttcttgctcagactcttgagatcttggcaggcaaatcgaaggatgaggcgatgaatgactacatcaccaaggtgaagcagctccaggaggaggctgccgcctcttaaaagtctcttaatagtgttgccaaacagctatttgtcagatgagctttgtgccatttcctcattagaagtatatcattcgatccattggggatctgtgaaccatgtacctgtttatgtaacttttttttttttcgaaagagcgcaggaggactgcgcacccatatattaaagtagagggaaaataaagggccataatgatgatatccatctatctatctatgtaactggtttatgtaccatgtacctgtttatgtaactggttaccagtgttatccatctatctatctatctgtgaagacttgagtcttctaaattatgatatctgtctgtgtgatatctgaggctgtgtttcgttttacaggctagtttggcttctactgttttcacaatgtttttgtctctatagctttttgaaagtgtttctactgttaaaacgtggaaactcaaccaagcggttagaatccgaagctggtttttcaaaagctggcgctttcccgtagcctgcttttggctttcagttttgttttttttttttccaaatggatgaaaatacagatatgtttttacaactgtaccaagcaagagaaagagaaagataaaagagata

SEQ ID NO: 7 - DNA, 276 bp, Zea mays
atgggtctgcaggaggagttcgaggagcacgccgagaaggccaagaccctgcctgccaccacgacgaacgagagcaagctcgtcctctacggcctctacaagcaggccaccgtcggcgacgtcagcaccggtcgtcctggcatcttcagcctgaaagagagagccaaatgggatgcttggaaggctgttgaagggaaatccaaggacgaagcaatggccgactacatcaccaaggtgaagcagctgctggaggccgccgccgcatccacctcctag

SEQ ID NO: 8 - protein, 91 aa, Zea mays
MGLQEEFEEHAEKAKTLPATTTNESKLVLYGLYKQATVGDVSTGRPGIFSLKERAKWDAWKAVEGKSKDEAMADYITKVKQLLEAAAASTS

SEQ ID NO: 9 - DNA, 1081 bp, Zea mays
agaagagcaagcagtaacgcgggaagcgagggggggcgggcccacggacaaaacagtggatagatataaacaagagggcgagcgcatcagctcggacttccccgccccgtccaagagcagcgccgtctcgtcgccatgggtctgcaggtacggcttctctcgccgctctgtatttgtatctctacctgcatcttcgaacaagtgccgtgtttgggttgtggagcaaaacctgctgtgtggtttaatttgtgacctcgccgtcgccggccggttcggttgttgcttgcttggtggcaacaagctcagtctctctggattaggtgggatggtaccgcccagcgtcgtcgctgatgtatggatggagtccgtggttcaggttccatccagagccaacgagacggtagcgcatcgccgtcgatgccggcaacgccatccgatccttgcttttatgggttgcgtttgctagccttctctctgattttggcaggaggagttcgaggagcacgccgagaaggccaagaccctgcctgccaccacgacgaacgagagcaagctcgtcctctacggcctctacaagcaggccaccgtcggcgacgtcagcaccggtacgtttgcgtttttttttggtctcgtcgatctccaatctcctgcctgctgctgctcgggatgttttctacaagtattagttccaataatctcgtcactctactctgaacctagctgttctgtctctgctaggtcgtcctggcatcttcagcctgaaagagagagccaaatgggatgcttggaaggctgttgaagggaaatccaaggacgaagcaatggccgactacatcaccaaggtgaagcagctgctggaggccgccgccgcatccacctcctaggctactcaagggaaaacaaaaaccatgatccgcagtcgcatatacgtatacatgttgtcatgatatacgctcgccaataaatcttatcattgttgtactgcttggtatggcttctgatgccatatatactcatcacccttcaaactaaggctgtaaactcttgcgaaaacaaactggatgtccctgtgatactagca

SEQ ID NO: 10 - DNA, 20 bp, Artificial Sequence
  Oligonucleotide
tgtcaatactgctcgtcctg

SEQ ID NO: 11 - DNA, 20 bp, Artificial Sequence
  Oligonucleotide
tagtcgctcattgcttcctc

SEQ ID NO: 12 - DNA, 21 bp, Artificial Sequence
  Oligonucleotide
atgggtttgcaggaggagttt

SEQ ID NO: 13 - DNA, 21 bp, Artificial Sequence
  Oligonucleotide
ctgcttgtagaggccatagag

SEQ ID NO: 14 - DNA, 21 bp, Artificial Sequence
  Oligonucleotide
tgggtctgcaggaggattttg

SEQ ID NO: 15 - DNA, 21 bp, Artificial Sequence
  Oligonucleotide
acggtggcctgcttgtagagt

SEQ ID NO: 16 - DNA, 20 bp, Artificial Sequence
  Oligonucleotide
gcatctggctgctggtgtag

SEQ ID NO: 17 - DNA, 22 bp, Artificial Sequence
  Oligonucleotide
gcatttgcattgacaagaatct

SEQ ID NO: 18 - DNA, 20 bp, Artificial Sequence
  Oligonucleotide
gaggctattccaggatggat

SEQ ID NO: 19 - DNA, 22 bp, Artificial Sequence
  Oligonucleotide
ctgtcatgttggttgattgtat

SEQ ID NO: 20 - DNA, 20 bp, Artificial Sequence
  Oligonucleotide
ggtggtggcaataacaaaag

SEQ ID NO: 21 - DNA, 19 bp, Artificial Sequence
  Oligonucleotide
gcaaggggaacacgacctt

SEQ ID NO: 22 - DNA, 20 bp, Artificial Sequence
  Oligonucleotide
aggccgtcctctctctgtat

SEQ ID NO: 23 - DNA, 20 bp, Artificial Sequence
  Oligonucleotide
ggggagagcatatccttcat

SEQ ID NO: 24 - DNA, 29 bp, Artificial Sequence
  Oligonucleotide
tagctgcagtaatgtcctgatgcgttgcg

SEQ ID NO: 25 - DNA, 28 bp, Artificial Sequence
  Oligonucleotide
tagggatcccgatggcgtctcgttctcg

SEQ ID NO: 26 - DNA, 27 bp, Artificial Sequence
  Oligonucleotide
gtcattttgtcaatgtgatgaccaggt

SEQ ID NO: 27 - DNA, 27 bp, Artificial Sequence
  Oligonucleotide
acctggtcatcacattgacaaaatgac

SEQ ID NO: 28 - DNA, 29 bp, Artificial Sequence
  Oligonucleotide
acgagatgtcatcctgtactgtccatgtt

SEQ ID NO: 29 - DNA, 29 bp, Artificial Sequence
  Oligonucleotide
aacatggacagtacaggatgacatctcgt

SEQ ID NO: 30 - DNA, 29 bp, Artificial Sequence
  Oligonucleotide
tgtacatcagcgcaaatgacataaaagga

SEQ ID NO: 31 - DNA, 29 bp, Artificial Sequence
  Oligonucleotide
tccttttatgtcatttgcgctgatgtaca

SEQ ID NO: 32 - DNA, 30 bp, Artificial Sequence
  Oligonucleotide
atagcctcagtttgtttgtcatccctatat

SEQ ID NO: 33 - DNA, 30 bp, Artificial Sequence
  Oligonucleotide
atatagggatgacaaacaaactgaggctat

SEQ ID NO: 34 - DNA, 29 bp, Artificial Sequence
  Oligonucleotide
tagctgcagagttcactgttcctccctca

SEQ ID NO: 35 - DNA, 29 bp, Artificial Sequence
  Oligonucleotide
tagctgcagatgattttcggcccttaccg

SEQ ID NO: 36 - DNA, 29 bp, Artificial Sequence
  Oligonucleotide
tagctgcagatcgccacacacgagaggct

SEQ ID NO: 37 - DNA, 29 bp, Artificial Sequence
  Oligonucleotide
tagctgcagggctcaaaagattcgtctcg

SEQ ID NO: 38 - DNA, 25 bp, Artificial Sequence
  Oligonucleotide
caatcccactatccttcgcaagacc

(19) United States
(12) Patent Application Publication
(10) Pub. No.: US 2021/0180078 A1
(43) Pub. Date: Jun. 17, 2021

(54) TRANSGENIC RICE PLANTS OVEREXPRESSING ACYL-COA-BINDING PROTEIN2 SHOW ENHANCED GRAIN SIZE

(71) Applicant: The University of Hong Kong, Pokfulam (HK)

(72) Inventors: Mee Len Chye, Pokfulam (HK); Ze Hua Guo, Kennedy Town (HK)

(21) Appl. No.: 16/768,319

(22) PCT Filed: Nov. 29, 2017
(86) PCT No.: PCT/CN2017/113489

§ 371 Date: May 29, 2020

(51) Int. Cl. C12N 15/82; C07K 14/415

(52) U.S. Cl. CPC C12N 15/8247; C07K 14/415; C12N 15/8261; C12N 15/8234

(57) ABSTRACT

The present disclosure provides a transgenic plant, seed or progeny genetically engineered to overexpress one or more exogenous Oryza sativa acyl-CoA-binding protein 2 (OsACBP2) in an amount effective to enhance grain size and/or weight relative to a vector-transformed control plant. Also provided are methods of enhancing grain size and/or weight by genetically engineering a plant to overexpress one or more exogenous OsACBP2 in an amount effective to enhance grain size and/or weight relative to a vector-transformed control plant. In certain embodiments the plant belongs to the Poaceae family.

### INCORPORATION OF SEQUENCE LISTING

The sequence listing that is contained in the file named “VRST004WO_ST25.txt”, which is 17 kilobytes (as measured in Microsoft Windows®) and was created on Nov. 9, 2017, is filed herewith by electronic submission and is incorporated by reference herein.

### FIELD OF THE INVENTION

The present disclosure relates generally to the field of plant engineering. In particular, the present disclosure relates to genetically engineered plants that overexpress Oryza sativa acyl-CoA-binding protein 2 (OsACBP2) in an amount effective to enhance grain size and/or weight.

### BACKGROUND OF THE INVENTION

Rice is considered the most important food crop, acting as a daily necessity for more than three billion people globally, and of all cereals, rice has the second largest production after maize. In 2014, rice was cultivated on ˜162 million hectares, and in 2016/2017, over 43 million tons were traded worldwide. From 1966 to 2000, rice production rose by 130%, but to satisfy the expansion of the global population, it must be further enhanced to 852 million tons by 2035 (Khush, Plant Breeding 132:433-436, 2013).

To this end, genes that improve grain size and yield of this staple crop have been intensively sought. One such example is the quantitative trait locus (QTL) for Grain Width on Chromosome 2 (GW2) which regulates endosperm formation and determines grain width and length as well as grain weight (Song. et al., Nat. Genet. 39:623-630, 2007). A nearly isogenic line (NIL), NIL(GW2), was demonstrated to enhance grain width by 26.2%, thickness (10.5%), and length (6.6%). GW7 was mapped to the same region as a QTL for Grain Length on Chromosome 7 (GL7), and participated with OsSPL16 (GW8) in grain size regulation (Wang, et at, Nat. Genet. 47:949-955, 2015), while OsSPL13 RNAi lines displayed decline in both grain length (˜10%) and grain weight (˜10%) (Si, et al., Nat. Genet. 48:447-456, 2016). While the NIL(GL7) line showed improvement in grain length (˜10%), grain width decreased by ˜10%. The overexpression of a QTL for Grain Size on Chromosome 5 (GS5) in transgenic rice also increased grain width (˜10%) (Li, et al., Nat. Genet. 43:1266-U1134, 2011). By contrast, deletion of GW5 caused gain in grain weight (˜20%), while knockout of ORF3 in GW5 increased seed width by ˜30% (Shomura, et al., Nat. Genet. 40:1023-1028, 2008; Liu, et al., Nat. Plants 3:17043, 2017). The overexpression of GS3 in transgenic rice led to reduction in grain length (˜20%) but rise in grain width (˜10%), culminating in a ˜30% reduction in grain weight (Mao, et al., Proc. Natl. Acad. Sci. USA 107:19579-19584, 2010).

Although acyl-CoA binding proteins (ACBPs), which show conservation at the acyl-CoA-binding (ACB) domain, have been reported to confer tolerance to stress and play various roles in plant development including embryogenesis, there have been no reports on any single ACBP that can confer gain in seed size and weight when overexpressed in transgenic plants.

In Arabidopsis and rice, six ACBPs co-exist in each species and they fall into four classes. Class I ACBPs are small (10 kDa), and include Arabidopsis AtACBP6 and rice OsACBP1, OsACBP2 and OsACBP3 (Meng, et al., New Phytol. 190:807-807, 2011). Interestingly, three such members (OsACBP1, OsACBP2, and OsACBP3) are present in rice, in comparison to only one in Arabidopsis. The 10-kDa ACBPs are the most common ACBP and only consist of a single ACB domain (Burton, et al., Biochem. 1 392:299-307, 2005). Unlike OsACBP1 and OsACBP2, OsACBP3 which shows 94% amino acid homology to OsACBP1 at the ACB domain, possesses a 63-amino-acid extension at its C-terminus. AtACBP6 was verified to be expressed in seeds using AtACBP6pro::GUS-transformed Arabidopsis (Hsiao, et al., Biosci. Rep. 34:865-877, 2014), consistent with microarray data from the Arabidopsis eFP browser. Using quantitative RT-PCR (qRT-PCR), OsACBP2 was more highly expressed in milk and soft dough seeds than OsACBP1 or OsACBP3 (Meng, et al., 2011, supra).

Each Class II ACBP has a transmembrane domain and an ankyrin-repeat domain. AtACBP1 and AtACBP2 were found to be abundantly expressed in seeds at various developmental stages by immunolocalization, GUS staining of AtACBPpro::GUS transgenic lines, and western blot analysis, again consistent with microarray analysis, however AtACBP1- and AtACBP2-OE lines have not been reported to produce seeds that display gain in size and weight. Class III OsACBP5 was expressed in milk and soft dough seeds on qRT-PCR analysis, and it was further suggested to be highly expressed in palea and lemma which are localized outside the seeds by microarray data. Class IV Kelch motif-containing AtACBP4 and AtACBP5 were reported to be expressed in seeds by microarray analysis, qRT-PCR, and GUS staining of AtACBPpro::GUS-transformed Arabidopsis.

Therefore, identification of a gene that when overexpressed produces increased grain size and weight in rice would represent a significant advance in the art.

### SUMMARY OF THE INVENTION

The present disclosure provides a method of increasing grain size, weight, or lipid content in grain bran or whole seed, in a plant, comprising upregulating expression of an acyl-CoA-binding protein 2 gene in the plant, wherein the grain size, weight, or lipid content in grain bran or whole seed of the plant is increased when compared to a control plant that lacks the upregulating. Thus the present disclosure additionally provides a plant, or a part thereof, produced by the method of increasing grain size, weight, or lipid content in grain bran or whole seed, in a plant, comprising upregulating expression of an acyl-CoA-binding protein 2 gene in the plant, wherein the grain size, weight, or lipid content in grain bran or whole seed of the plant is increased when compared to a control plant that lacks the upregulating. In certain embodiments the upregulating comprises introducing into the plant a recombinant nucleic acid construct comprising the acyl-CoA-binding protein 2 gene operably linked to a heterologous promoter.

In some embodiments the acyl-CoA-binding protein 2 gene is an Oryza sativa acyl-CoA-binding protein 2 gene. In particular embodiments, the acyl-CoA-binding protein 2 gene comprises a polynucleotide sequence having at least 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity to SEQ ID NO: 1. In other embodiments, the acyl-CoA-binding protein 2 gene comprises the polynucleotide sequence of SEQ ID NO:1, SEQ ID NO:4 or SEQ ID NO:7. In other embodiments, the acyl-CoA-binding protein 2 gene encodes a polypeptide having at least 82%, 83%, 84%, 85%, 86%, 87%, 88%, 89%, 90%, 91%, 92%, 93%, 94%, 95%, 96%, 97%, 98%, 99% or greater sequence identity to SEQ ID NO:2. In still other embodiments the acyl-CoA-binding protein 2 gene encodes the polypeptide sequence of SEQ ID NO:2, SEQ ID NO:5 or SEQ ID NO:8. In additional embodiments the heterologous promoter is a constitutive, tissue-specific or inducible promoter.

In certain embodiments the grain size of the plant is increased, for example by about 5%, about 10%, about 15%, about 20% or more compared to grain from a control plant that lacks the upregulating. In other embodiments the grain weight of the plant is increased, for example by about 5%, about 10% or more compared to grain from a control plant that lacks the upregulating. In further embodiments the lipid content in grain bran of the plant is increased, for example by about 5%, about 10% or more compared to grain bran from a control plant that lacks the upregulating. In yet further embodiments the lipid content in whole seed of the plant is increased, for example by about 5%, about 10% or more compared to whole seed from a control plant that lacks the upregulating.

In some embodiments the plant is a monocotyledonous plant, for example a plant in the Poaceae family, such as a rice plant. In particular embodiments the plant is an Oryza sativa plant. In other embodiments the plant is a dicotyledonous plant.

The present disclosure also provides a plant comprising a recombinant nucleic acid construct comprising a polynucleotide sequence having at least 77% sequence identity to SEQ ID NO:1, or a polynucleotide sequence encoding a polypeptide having at least 82% sequence identity to SEQ ID NO:2, wherein the polynucleotide sequence is operably linked to a heterologous promoter, and wherein the grain size, weight, or lipid content in grain bran or whole seed, of the plant is increased when compared to a plant that lacks the recombinant nucleic acid construct. Therefore, the present disclosure additionally provides a seed, plant part or progeny of a plant comprising a recombinant nucleic acid construct comprising a polynucleotide sequence having at least 77% sequence identity to SEQ ID NO:1, or a polynucleotide sequence encoding a polypeptide having at least 82% sequence identity to SEQ ID NO:2, wherein the polynucleotide sequence is operably linked to a heterologous promoter, and wherein the grain size, weight, or lipid content in grain bran or whole seed, of the plant is increased when compared to a plant that lacks the recombinant nucleic acid construct, wherein the seed, plant part or progeny comprises the recombinant nucleic acid construct.

The present disclosure further provides a method of producing grain having increased size, weight, or lipid content in grain bran or whole seed, the method comprising obtaining a plant comprising upregulated expression of an acyl-CoA-binding protein 2 gene in the plant, wherein the size or weight of the grain, or the lipid content in grain bran or whole seed is increased when compared to a plant that lacks the upregulated expression, growing the plant under plant growth conditions to produce grain from the plant, and collecting grain from the plant. In certain embodiments collecting grain from the plant comprises harvesting the grain from the plant. In some embodiments the plant is a rice plant. In particular embodiments the rice plant is an Oryza sativa plant.

The present disclosure additionally provides a method of producing a plant having increased grain size, weight, or lipid content in grain bran or whole seed, comprising crossing a plant comprising a recombinant nucleic acid construct comprising a polynucleotide sequence having at least 77% sequence identity to SEQ ID NO:1, or a polynucleotide sequence encoding a polypeptide having at least 82% sequence identity to SEQ ID NO:2, wherein the polynucleotide sequence is operably linked to a heterologous promoter, and wherein the grain size, weight, or lipid content in grain bran or whole seed, of the plant is increased when compared to a plant that lacks the recombinant nucleic acid construct with a second plant to produce at least a first progeny plant comprising increased grain size, weight, or lipid content in grain bran or whole seed, when compared to a control plant that lacks the recombinant nucleic acid construct. In some embodiments the plant is a rice plant, for example an Oryza sativa rice plant.

The present disclosure also provides a method of increasing grain size, weight, or lipid content in grain bran or whole seed, in a rice plant, comprising introducing into the rice plant a recombinant nucleic acid construct comprising a polynucleotide sequence having at least 77% sequence identity to SEQ ID NO:1, or a polynucleotide sequence encoding a polypeptide having at least 82% sequence identity to SEQ ID NO:2, wherein the polynucleotide sequence is operably linked to a heterologous promoter, and wherein the grain size or weight, or lipid content in grain bran or whole seed of the rice plant is increased when compared to a rice plant that lacks the recombinant nucleic acid construct.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1. OsACBP2 is highly expressed in the embryos and endosperm of germinating wild-type rice seeds. Relative expression of OsACBP1 (Locus ID Os08g0162800; first two bars in each set), OsACBP2 (Locus ID Os06g0115300; second two bars in each set), OsACBP3 (Locus ID Os03g0576699; third two bars in each set), OsACBP4 (Locus ID Os04g0681900; fourth two bars in each set), OsACBP5 (Locus ID Os03g0243600; fifth two bars in each set), and OsACBP6 (Locus ID Os03g0835600; sixth two bars in each set) from rice Zhonghua11 wild-type seeds at different days after germination is shown. On each day after imbibition, embryos (open bars) and endosperm (striped bars) were dissected from ten seeds. Gene expression of OsACBP1, OsACBP2, OsACBP3, OsACBP4, OsACBP5, and OsACBP6 was examined by quantitative RT-PCR on a StepOne Plus Real-time PCR system using SYBR Green Mix (Applied Biosystems) performed with gene-specific primers (Table 1). The relative mRNA amount was normalized against the expression of rice ACTIN. H and L indicate higher and lower expression respectively in comparison to expression one day after germination using the Student's t-test (P<0.05, n=3).

FIG. 2A, FIG. 2B, FIG. 2C, FIG. 2D, FIG. 2E, FIG. 2F, FIG. 2G, FIG. 2H and FIG. 2I. OsACBP2pro::GUS is expressed in developing and germinating rice seeds in bright-field light microscopy. Histochemical β-glucuronidase (GUS) staining shows OsACBP2pro::GUS in developing transgenic rice seeds 5 days after fertilization (DAF) (FIG. 2A), 10 DAF (FIG. 2B), 15 DAF (FIG. 2C), 16 DAF (FIG. 2D), and imbibed mature rice seeds (FIG. 2E). In imbibed mature rice seeds, staining of the endosperm (FIG. 2F), embryo (FIG. 2G), aleurone layer (FIG. 2H) and scutellum (FIG. 2I) is displayed. Bars=1 mm (FIG. 2A-FIG. 2E) or 50 μm (FIG. 2F-FIG. 2I). a, aleurone layer, e, embryo, s, starchy endosperm, sc, scutellum. Boxed regions in (FIG. 2F and FIG. 2G) are magnified in (FIG. 2H and FIG. 2I), respectively.

FIG. 3A, FIG. 3B and FIG. 3C. Regulation of OsACBP2 expression by Skn-I-like motifs in 1-d-old germinating rice seeds. FIG. 3A. Schematic diagram of OsACBP2pro::GUS constructs in which the OsACBP2 5′-flanking sequence was fused to the GUS reporter gene. Putative cis-elements on the OsACBP2 5′-flanking sequence were predicted using PlantCARE. Putative cis-elements on the OsACBP2 5′-flanking region (pOS806) and its deletion derivatives (pOS883, pOS837, pOS886 and pOS859) are presented by rectangles (Skn-I-like motifs), triangles (TGACG motifs), and ovals (ABRE motifs). The plasmid DX2181 was used as a vector control. Skn-I, Skn-I-like motif; TGACG, TGACG motif binding factor; ABRE, abscisic acid-responsive element; GUS, β-glucuronidase. The putative Skn-I-like motifs corresponded to −1486/−1482, −956/−952 and −939/−935, −826/−822 and −766/−762. The putative TGACG motifs were at −1271/−1267, −1189/−1185, −339/−335, and −33/−29. A putative ABRE was predicted at −157/−147. Numbers are relative to the transcription start site. FIG. 3B. Histochemical β-glucuronidase (GUS) staining (Jefferson, et al., EMBO J. 6:3901-3907, 1987) of 1-d-old seeds from transgenic ZH11 OsACBP2pro::GUS rice transformants of pOS806 and its deletion derivatives (pOS883, pOS837, pOS886 and pOS859). The DX2181 transformant (DX2181) was used as a vector control. Bars=1 mm. FIG. 3C. Electrophoretic mobility shift assays using LightShift® Chemiluminescent EMSA Kit (ThermoFisher Scientific) show the binding of nuclear extracts to the Skn-I-like motifs in the OsACBP2 5′-flanking region. The competitor contains the 200× non-biotin-labeled probe. −, unavailable in the reaction; +, available in the reaction. Arrowheads indicate the DNA-protein binding complexes formed in the presence of nuclear proteins extracted from 1-d-old rice seeds. An arrow indicates the free unbound biotin-labeled probe. Primers ML2564/ML2565 were used to generate probes containing Skn-I-like motif at −1486/−1482; ML2523/ML2524 for −956/−952 and −939/−935; ML2567/ML2568 for −826/−822; and ML2525/ML2526 for −766/−762 (Table 1).

FIG. 4A, FIG. 4B and FIG. 4C. Characterization of four rice osacbp2 mutants, RMD_03Z11AZ19, RMD_03Z11LE18, RMD_03Z11LG76, and PFG_1D-05815. FIG. 4A. Schematic representation of the genomic structure of OsACBP2 (Os06g0115300). Open boxes indicate the 5′- or 3′-untranslated regions (UTRs), filled boxes represent exons (numbered in Roman numerals) and lines between them represent introns. Locations of T-DNA or retrotransposon insertions in the putative mutants (PFG_1D-05815, RMD_03Z11AZ19, RMD_03Z11LE18 and RMD_03Z11LG76) are denoted using triangles. FIG. 4B. Western blot analysis of OsACBP2 expression in wild-type Zhonghua11 and Hwayoung, and putative osacbp2 mutants. Anti-OsACBP1 antibodies have been shown to cross-react with OsACBP2 as OsACBP1 shares 79% amino acid sequence identity to OsACBP2. Twenty μg of total seed proteins were separated on a 15% SDS-PAGE. An arrowhead indicates the 10-kDa OsACBP2 cross-reacting band. FIG. 4C. The growth of rice seeds at 1 to 4 days after germination (DAG). For each group, six seeds were tested in each of three repeats, and the results were consistent. Bar=1 cm.

FIG. 5A, FIG. 5B, FIG. 5C, FIG. 5D, FIG. 5E, FIG. 5F, FIG. 5G, FIG. 5H, FIG. 5I, FIG. 5J and FIG. 5K. Phenotypic analysis of 2-week-old OsACBP2-overexpressing lines (OsACBP2-OE, OE-1, OE-3, OE-17 and OE-21; 35spro::OsACBP2) and osacbp2 (AZ19, LE18, LG76 and P05815) rice seedlings. Growth of transgenic OsACBP2-OE and osacbp2 rice seedlings. Thirty plants for each rice line were germinated and grown in rice growth medium (Lam, et al., Plant Physiol. pp. 00566, 2015). FIG. 5A, FIG. 5B, FIG. 5C and FIG. 5D. OsACBP2-OE lines (OE-1, OE-3, OE-17 and OE-21). FIG. 5E. pCXSN vector control (Chen, et al., Plant Physiol. 150:1111-1121, 2009) for OsACBP2-OE. FIG. 5F. Zhonghua11 wild-type. FIG. 5G. Hwayoung wild-type. FIG. 5H, FIG. 5I and FIG. 5J. osacbp2 mutants (RMD_03Z11LE18, RMD_03Z11LG76 and RMD_03Z11AZ19), derived from wild-type Zhonghua11. FIG. 5K. osacbp2 PFG_1D-05815, derived from wild-type Hwayoung. Bars=1 cm.

FIG. 6A, FIG. 6B, FIG. 6C and FIG. 6D. Measurement of grain length, width and weight in OsACBP2-OEs and osacbp2 mutants as described in Liu, et al., Nat. Plants 3:17043, 2017. FIG. 6A. Rice grains from pCXSN vector control (VC), OsACBP2-OEs (OE-1, OE-3, OE-17, and OE-21), Zhonghua11 wild-type (ZH11), osacbp2 RMD_03Z11AZ19 (AZ19), osacbp2 RMD_03Z11LE18 (LE18), osacbp2 RMD_03Z11LG76 (LG76), Hwayoung wild-type (HY), and osacbp2 PFG_1D-05815 (P05815). The background for AZ19, LE18 and LG76 is ZH11, while that for P05815 is HY. Bar=10 mm. Comparison in grain length (FIG. 6B), grain width (FIG. 6C), and grain weight (FIG. 6D) amongst the OsACBP2-OEs (OE-1, OE-3, OE-17, and OE-21), controls (VC, ZH11, and HY), and osacbp2 mutants (P05815, AZ19, LE18, LG76) (n=30). P values from the Student's t-test of the OsACBP2-OEs and osacbp2 mutants in comparison to their controls are indicated. The values in FIG. 6B, FIG. 6C and FIG. 6D represent the mean±S.E.

FIG. 7A, FIG. 7B, FIG. 7C, FIG. 7D, FIG. 7E, FIG. 7F, FIG. 7G, FIG. 7H, FIG. 7I, FIG. 7J and FIG. 7K. Transmission electron microscopy (TEM) of the scutellum cells from imbibed transgenic rice seeds from OsACBP2-overexpressing (OsACBP2-OE) and osacbp2 mutants. Scutellum cells of OsACBP2-OEs, OE-1 (FIG. 7A), OE-3 (FIG. 7B), OE-17 (FIG. 7C), and OE-21 (FIG. 7D), showed increase in oil bodies and protein bodies in comparison to the pCXSN vector control (FIG. 7E). Scutellum cells in the osacbp2 mutant lines AZ19 (FIG. 7H), LE18 (FIG. 7I), LG76 (FIG. 7J), and P05815 (FIG. 7K), contained fewer oil bodies compared to the ZH11 wild-type (FIG. 7F) and Hwayoung wild-type (FIG. 7G). Oil bodies are indicated by arrowheads. Bars=2 μm.

FIG. 8. Gas chromatography-mass spectrometry analysis of fatty acids from OsACBP2-OE transgenic rice embryos. Quantitative analysis of fatty acids (C14:0, C16:0, C16:1, C18:0, C18:1, C18:2, C18:3, C20:0, C20:1 and C22:0-FA) from embryos of rice OsACBP2-OE, OE-1 (first bar in each set), OE-3 (second bar in each set), OE-17 (third bar in each set), OE-21 (fourth bar in each set), pCXSN vector-transformed control (fifth bar in each set) and ZH11 wild-type (sixth bar in each set). Values are mean±S.E. of measurements made on three independent batches of samples. Student's t test for *, P<0.05. Each measurement contains 40 seeds.

FIG. 9A, FIG. 9B, and FIG. 9C. Protein content and composition in mature rice seeds from OsACBP2-OEs. FIG. 9A. Protein content measurements in embryos and endosperm from imbibed rice seeds of OsACBP2-OEs (OE-1, OE-3, OE-17, and OE-21). Wild-type (WT) and pCXSN vector control (VC) were used as controls. FIG. 9B. Coomassie blue-stained 10% SDS-PAGE shows 30 μg of total embryo protein from OsACBP2-OEs and controls. An arrowhead indicates the putative 46-kDa rice storage globulin. FIG. 9C. Coomassie blue-stained 10% SDS-PAGE shows 30 μg of total endosperm protein from OsACBP2-OEs and controls. An arrowhead indicates the 55-kDa 11S glutelin band. Brackets denote acidic chains (28-31 kDa) and basic chains (20-22 kDa).

FIG. 10. Microarray analysis showing the relative expression levels of six OsACBPs in various organs. Microarray data from RiceXPro (Sato, et al., Nucl. Acids Res. 39:D1141-D1148, 2010) showed relative mRNA levels of six OsACBPs, OsACBP1 (Locus ID Os08g0162800), OsACBP2 (Locus ID Os06g0115300), OsACBP3 (Locus ID Os03g0576699), OsACBP4 (Locus ID Os04g0681900), OsACBP5 (Locus ID Os03g0243600), and OsACBP6 (Locus ID Os03g0835600) in various organs of wild-type rice at normal condition. Light shading indicates high expression, and dark shading indicates low expression.

FIG. 11. Transgenic Arabidopsis AtACBP6-OE lines and the atacbp6 mutant do not differ in seed size from the Col-0 wild-type. The projective size of transgenic Arabidopsis AtACBP6-OEs (OE-3 and OE-5) and atacbp6 mature seeds in comparison to the Col-0 wild-type (WT) (n=1000) was measured and calculated as described in Herridge, et al., Plant Methods 7:3, 2011. P values from the Student's t-test of the AtACBP6-OEs and atacbp6 mutant in comparison to WT were indicated. The values represent the mean±S.D.

FIG. 12. Growth rates of 2-day-old (top panel) and 2-week-old (bottom panel) transgenic OsACBP2-OE and osacbp2 rice seedlings. Coleoptile elongation rate was measured 2 days after germination and shoot elongation rate was measured 2 weeks after germination. Black bars indicate control lines; white OE lines; and striped osacbp mutants. Values are mean±S.E. of measurements made on ten plants. Asterisks indicate significant difference by the Student's t test (P<0.05).

### DETAILED DESCRIPTION OF THE INVENTION

The following detailed description is provided to better define the present disclosure and to guide those of ordinary skill in the art in the practice of the present disclosure. Unless otherwise noted, terms are to be understood according to conventional usage by those of ordinary skill in the relevant art.

Acyl-CoA-binding proteins (ACBPs) are a family of proteins that facilitate the binding of long-chain acyl-CoA esters at a conserved acyl-CoA-binding domain. ACBPs act to form intracellular acyl-CoA pools, transport acyl-CoA esters and regulate lipid metabolism. In the model plant Arabidopsis thaliana, a family of six ACBPs has been demonstrated to function in stress and development. Six ACBPs (OsACBPs) have also been identified in Oryza sativa (rice), but they are not as well characterized as those in Arabidopsis thaliana.

As rice seeds provide food for more than three billion people worldwide, the identification of rice genes that enhance grain size and composition is needed to satisfy the demands of a growing global population. Herein, Oryza sativa (rice) ACBP2 was demonstrated not only important in seed development and germination, but also enhanced grain size by ˜20%. When OsACBP2 function was investigated using osacbp2 mutants and transgenic rice overexpressing OsACBP2 (OsACBP2-OE), osacbp2 was retarded during germination, while OsACBP2-OEs were enhanced in grain size and/or weight as well as seedling growth. Transmission electron microscopy detected the accumulation of oil bodies in the OsACBP2-OE scutellum cells, while gas chromatography-mass spectrometry of mature OsACBP2-OE seeds revealed ˜10% gains in C18:1- and C18:2-fatty acids over the vector-transformed control; these changes likely attributed to gain in grain size. As fatty acids are predominantly stored in the rice bran, rather than the starchy endosperm in rice seeds, and dietary rice bran contains beneficial bioactive components, OsACBP2 represents a promising candidate for enriching seed nutritional value. OsACBP2 is the first ACBP to be demonstrated to confer increases in grain size viz. seed length (˜10%), seed width (˜10%), and seed weight (˜10%) accompanied by enhancement in lipid content (˜10%) in whole seed.

The present disclosure provides a transgenic plant, seed and progeny thereof genetically engineered to overexpress one or more exogenous Oryza sativa acyl-CoA-binding protein 2 (OsACBP2) coding sequences in an amount effective to enhance grain size as compared to a vector (pCXSN)-transformed control plant. The transgenic plant belongs to the Poaceae family. Also provided is a plant product, e.g., a commodity product, derived from the transgenic plant, which product overexpresses the one or more exogenous OsACBP2.

The present disclosure also provides a method of enhancing grain size and/or weight. Such method comprises genetically engineering a plant to overexpress one or more exogenous OsACBP2 coding sequences in an amount effective to enhance grain size relative to a vector-transformed control plant. In one embodiment, the method comprises the steps of transforming a plant with a vector comprising one or more exogenous nucleic acid sequences encoding the one or more exogenous OsACBP2 polypeptide operably linked to one or more plant expressible promoter; and expressing the one or more exogenous OsACBP2 in the plant in an amount effective to provide enhanced grain size relative to a vector-transformed control plant. In still another embodiment, the one or more plant expressible promoter is selected from the group consisting of a constitutive promoter, a tissue-specific promoter and an inducible promoter. In one embodiment, the plant belongs to the Poaceae family.

### Nucleic Acids, Polypeptides and Plant Transformation Constructs

Certain embodiments of the current disclosure concern the use of recombinant nucleic acid sequences comprising an OsACBP2 coding sequence. An exemplary nucleic acid sequence comprising an OsACBP2 coding sequence is provided as SEQ ID NO:1, an exemplary amino acid sequence encoded by an OsACBP2 coding sequence is provided as SEQ ID NO:2, and an exemplary genomic sequence encoding an OsACBP2 amino acid sequence is provided as SEQ ID NO:3. Complements to any nucleic acid sequences described herein can also be used in certain embodiments. Genes or cDNAs encoding OsACBP2 useful in the present disclosure include naturally occurring OsACBP2 (GenBank/EMBL data library under accession numbers Os06g0115300). Other genes useful for conferring enhanced grain size and/or weight to plants include variants of OsACBP2. For instance, an exemplary nucleic acid sequence comprising a Zea mays ZmACBP1 coding sequence is provided as SEQ ID NO:4, an exemplary amino acid sequence encoded by an ZmACBP1 coding sequence is provided as SEQ ID NO:5, and an exemplary genomic sequence encoding an ZmACBP1 amino acid sequence is provided as SEQ ID NO:6. An exemplary nucleic acid sequence comprising a Zea mays ZmACBP2 coding sequence is provided as SEQ ID NO:7, an exemplary amino acid sequence encoded by a ZmACBP2 coding sequence is provided as SEQ ID NO:8, and an exemplary genomic sequence encoding a ZmACBP2 amino acid sequence is provided as SEQ ID NO:9. In certain embodiments the coding sequence of the selected gene may be genetically engineered by altering the coding sequence for increased or optimal expression in the crop species of interest. Methods for modifying coding sequences to achieve optimal expression in a particular crop species are well-known (see, e.g., Perlak, et al., Proc. Natl. Acad. Sci. USA, 88: 3324, 1991; and Koziel, et al., Biotechnol. 11: 194, 1993).

In certain embodiments, nucleic acids are used that have at least about 77% (percent) sequence identity, about 78% sequence identity, about 79% sequence identity, about 80% sequence identity, about 81% sequence identity, about 82% sequence identity, about 83% sequence identity, about 84% sequence identity, about 85% sequence identity, about 86% sequence identity, about 87% sequence identity, about 88% sequence identity, about 89% sequence identity, about 90% sequence identity, about 91% sequence identity, about 92% sequence identity, about 93% sequence identity, about 94% sequence identity, about 95% sequence identity, about 96% sequence identity, about 97% sequence identity, about 98% sequence identity, or about 99% sequence identity to any of the nucleic acid sequences described herein. In other embodiments, polypeptides are used that have at least about 82% sequence identity, about 83% sequence identity, about 84% sequence identity, about 85% sequence identity, about 86% sequence identity, about 87% sequence identity, about 88% sequence identity, about 89% sequence identity, about 90% sequence identity, about 91% sequence identity, about 92% sequence identity, about 93% sequence identity, about 94% sequence identity, about 95% sequence identity, about 96% sequence identity, about 97% sequence identity, about 98% sequence identity, and about 99% sequence identity to any of the nucleic acid or protein sequences described herein. As used herein, the term “percent sequence identity” or “% sequence identity” refers to the percentage of identical nucleotides or amino acids in a linear polynucleotide or polypeptide sequence of a reference (“query”) sequence (or its complementary strand) as compared to a test (“subject”) sequence (or its complementary strand) when the two sequences are optimally aligned (with appropriate nucleotide or amino acid insertions, deletions, or gaps totaling less than 20 percent of the reference sequence over the window of comparison). Methods to determine “percent sequence identity” are codified in numerous publicly available programs including, but are not limited to, GCG (also known as The Wisconsin Package™), and the BLAST programs that are publicly available from NCBI. Optimal alignment of sequences for aligning a comparison window are well-known to those skilled in the art and may be conducted by tools including, but not limited to, the local homology algorithm of Smith and Waterman (Adv. Appl. Math. 2:482-489, 1981), the homology alignment algorithm of Needleman and Wunsch (J. Mol. Biol. 48:443-453, 1970), and the search for similarity method of Lipman and Pearson (Science 227:1435-1441, 1985).

The nucleic acids for use in the present disclosure may be from any source, e.g., identified as naturally occurring in a plant, or synthesized, e.g., by mutagenesis. In certain embodiments, the naturally occurring sequence may be from any plant. In some embodiments, the plant may be a dicotyledonous plant, for example, Arabidopsis, peanut (Arachis hypogaea), barrel medic (Medicago truncatula), carrot, soybean (Glycine max), cotton, Brassica, canola, tomato, potato, alfalfa, grape, clover, poplar, willow, eucalyptus, hemp, a Lotus sp., a Vinca sp., a Nicotiana sp., a Vitis sp., or a Ricinus sp. In other embodiments, the plant may be a monocotyledonous plant, for example maize, wheat, rice, sorghum (Sorghum bicolor), oats, barley, sugar cane, African oil palm (Elaeis guineensis), or switchgrass.

Coding sequences used in the present disclosure may be provided in a recombinant vector operably linked to a homologous or heterologous promoter functional in plants. Expression constructs may also be used comprising these sequences. In other embodiments, plants and plant cells transformed with the sequences may be provided. The construction of vectors which may be employed in conjunction with plant transformation techniques using these or other sequences according to the disclosure will be known to those of skill of the art in light of the present disclosure (see, for example, Sambrook, et al., Molecular Cloning: a Laboratory Manual, Volume 3, Cold Spring Harbor Laboratory Press, Cold Spring Harbor, N.Y., 1989). The techniques of the current disclosure are thus not limited to the use of any particular nucleic acid sequences.

The choice of any additional elements used in conjunction with the OsACBP2 coding sequences may depend on the purpose of the transformation. One of the major purposes of transformation of crop plants is to add commercially desirable, agronomically important traits to the plant, as described herein.

Vectors used for plant transformation may include, for example, plasmids, cosmids, YACs (yeast artificial chromosomes), BACs (bacterial artificial chromosomes) or any other suitable cloning system, as well as fragments of DNA therefrom. Thus when the term “vector” or “expression vector” is used, all of the foregoing types of vectors, as well as nucleic acid sequences obtained therefrom and otherwise, are included. It is contemplated that utilization of cloning systems with large insert capacities will allow introduction of large DNA sequences comprising more than one selected gene. In accordance with the disclosure, this could be used to introduce genes corresponding to, e.g., an entire biosynthetic pathway, into a plant.

Particularly useful for transformation are expression cassettes which have been derived from such vectors. DNA segments used for transforming plant cells will generally comprise the cDNA, gene or genes which one desires to introduce into and have expressed in the host cells. These DNA segments can further include structures such as promoters, enhancers, polylinkers, or even regulatory genes as desired. The DNA segment or gene chosen for cellular introduction will often encode a protein which will be expressed in the resultant recombinant cells resulting in a screenable or selectable trait and/or which will impart an improved phenotype to the resulting transgenic plant. Preferred components likely to be included with vectors used in the current disclosure are as follows.

A. Regulatory Elements

The selection of a promoter used in an expression cassette determines the spatial and temporal expression pattern of the transgene in the transgenic plant. Promoters vary in their strength, i.e., ability to promote transcription. Selected promoters express transgenes in specific cell types (such as leaf epidermal cells, mesophyll cells, root cortex cells) or in specific tissues or organs (such as roots, leaves or flowers), and the selection reflects the desired location of accumulation of the gene product. Alternatively, the selected promoter drives expression of the gene under various inducing conditions.

Various types of plant expressible promoters are suitable for the present disclosure, such as constitutive promoters, tissue-specific promoters and inducible promoters. Examples of suitable constitutive promoters for nuclear-encoded expression include, but are not limited to, the core promoter of the Rsyn7 promoter and other constitutive promoters disclosed in U.S. Pat. No. 6,072,050, incorporated herein by reference in its entirety. Other constitutive promoters for use in the present disclosure include, but are not limited to, rice actin (McElroy et al., Plant Cell 2:163-171, 1990); ubiquitin (Christensen, et al., Plant Mol. Biol., 12:619-632, 1989; and Christensen, et al., Plant Mol. Biol. 18:675-689, 1992); pEMU (Last et al., Theor. Appl. Genet. 81:581-588, 1991); MAS (Velten, et al., EMBO J., 3:2723-2730, 1984); and ALS promoter (U.S. Pat. No. 5,659,026, incorporated herein by reference in its entirety). Still other constitutive promoters are described in U.S. Pat. Nos. 5,608,149; 5,608,144; 5,604,121; 5,569,597; 5,466,785; 5,399,680; 5,268,463; 5,608,142, each of which is incorporated herein by reference in their entirety.

In certain embodiments, exemplary promoters for expression of a nucleic acid sequence include plant promoters such as the CaMV 35S (Odell, et al., Nature 313:810-812, 1985), CaMV 19S (Lawton, et al., Plant Mol. Biol. 9:315-324, 1987), nos (Ebert, et al., Proc. Natl. Acad. Sci. USA 84:5745-5749, 1987), actin (Wang, et al., Mol. Cell. Biol. 12:3399-3406, 1992), UDP glucose flavonoid glycosyl-transferase gene promoter (Ralston, et al., Genet. 119:185-197, 1988), MPI proteinase inhibitor (Cordero, et al., Plant J 6141-150, 1994), and the glyceraldehyde-3-phosphate dehydrogenase (Kohler, et al., Plant Mol. Biol. 29:1293-1298, 1995; Quigley, et al., J Mol. Evol. 29:412-421, 1989; Martinez, et al., J. Mol. Biol. 208:551-565, 1989) promoter, and the ubiquitin promoters from maize or rice, or ubiquitin promoters for use in various monocotyledonous plants (Christensen and Quail, Transgenic Res. 5:213-218, 1996).

Tissue-specific or tissue-preferred promoters can be used to target a gene expression within a particular tissue such as seed, leaf or root tissue. Tissue-specific promoters, such as Adh (Walker, et al., Proc. Natl. Acad. Sci. USA 84:6624-6628, 1987), sucrose synthase (Yang and Russell, Proc. Natl. Acad. Sci. USA 87:4144-4148, 1990), α-tubulin (Kim and An, Transgenic Research 1:188-194, 1992), cab (Sullivan, et al., Mol. Gen. Genet. 215:431-440, 1989), PEPCase (Hudspeth and Grula, Plant Mol. Biol. 12:579-589, 1989), lectin (Vodkin, et al., Cell 34:1023, 1983; Lindstrom, et al., Dev. Genet. 11:160, 1990), corn alcohol dehydrogenase 1 (Vogel, et al., J. Cell. Biochem. 13:Part D, 1989; Dennis, et al., Nucl. Acids Res. 12:3983-4000, 1984); corn light harvesting complex (Simpson, Science 233:34, 1986; Bansal, et al., Proc. Natl. Acad. Sci. USA 89:3654-3658, 1992), corn heat shock protein (Rochester, et al., EMBO J. 5:451-458, 1986), pea small subunit RuBP carboxylase (Poulsen, et al., Mol. Gen. Genet. 205:193-200, 1986; Cashmore, et al., Gen. Eng. of Plants, Plenum Press, New York, 29-38, 1983), Ti plasmid mannopine synthase or nopaline synthase (Langridge, et al., Proc. Natl. Acad. Sci. USA 86:3219-3223, 1989), petunia chalcone isomerase (Van Tunen, et al., EMBO J. 7:1257, 1988), bean glycine rich protein 1 (Keller, et al., EMBO J. 8:1309-1314, 1989), potato patatin promoters (Wenzler, et al., Plant Mol. Biol. 12:41-50, 1989), root cell promoters (Conkling, et al., Plant Physiol. 93:1203-1211, 1990), maize zein (Reina, et al., Nucl. Acids Res. 18:6426, 1990; Kriz, et al., Mol. Gen. Genet. 207:90-98, 1987; Wandelt and Feix, Nucl. Acids Res. 17:2354, 1989; Langridge and Feix, Cell 34:1015-1022, 1983; Reina, et al., Nucl. Acids Res. 18:6426, 1990), globulin-1 (Belanger and Kriz, Genet. 129:863-872, 1991), R gene complex-associated promoters (Chandler, et al., The Plant Cell 1:1175-1183, 1989), and chalcone synthase (Franken, et al., EMBO J. 10:2605-2612, 1991), or tissue selective promoters and tissue-specific enhancers (Fromm, et al., Nature 312:791-793, 1986, Fromm, et al., The Plant Cell 1:977-984, 1989) are also contemplated to be useful in certain embodiments.

Additional tissue-preferred promoters are described in Yamamoto, et al., Plant J. 12:255-265, 1997; Kawamata, et al., Plant Cell Physiol. 38:792-803, 1997; Hansen, et al., Mol. Gen. Genet. 254:337-343, 1997; Russell, et al., Transgenic Res. 6:157-168, 1997; Rinehart, et al., Plant Physiol. 112:1331-1341, 1996; Van Camp, et al., Plant Physiol. 112:525-535, 1996; Canevascini, et al., Plant Physiol. 112:513-524, 1996; Yamamoto, et al., Plant Cell Physiol. 35:773-778, 1994; Lam, Results Probl. Cell Differ. 20:181-196, 1994; Orozco, et al., Plant Mol. Biol. 23:1129-1138, 1993; Matsuoka, et al., Proc Natl. Acad. Sci. USA 90:9586-9590, 1993; and Guevara-Garcia, et al., Plant J. 4:495-505, 1993. Suitable tissue-specific expression patterns include green tissue specific, root specific, stem specific, and flower specific.

Promoters suitable for expression in green tissue include many that regulate genes involved in photosynthesis. Many of green tissue specific promoters have been cloned from both monocotyledons and dicotyledons, e.g., leaf-specific promoters that are known in the art (Kwon, et al., Plant Physiol. 105:357-367, 1994; Gotor, et al., Plant J. 3:509-518, 1993). Another example is the promoter encoding rbsC (Coruzzi, et al., EMBO J. 3:1671-1697, 1984).

Suitable root-preferred promoters may be selected from the ones known and widely available in the art or isolated de novo from various compatible species (see, e.g., Hire et al., Plant Mol. Biol. 20:207-218, 1992—soybean root-specific glutamine synthetase gene; Keller and Baumgartner, Plant Cell, 3:1051-1061, 1991—root-specific control element in the GRP 1.8 gene of French bean; Sanger, et al., Plant Mol. Biol. 14:433-443, 1990—root-specific promoter of the mannopine synthase (MAS) gene of Agrobacterium tumefaciens; Miao, et al., Plant Cell, 3:11-22, 1991—full-length cDNA clone encoding cytosolic glutamine synthetase (GS), which is expressed in roots and root nodules of soybean; also U.S. Pat. Nos. 5,837,876; 5,750,386; 5,633,363; 5,459,252; 5,401,836; 5,110,732; 5,023,179 and 7,285,656, each of which is specifically incorporated herein by reference in their entirety). Also, the Cauliflower Mosaic Virus (CaMV) 35S promoter has been reported to have root-specific and leaf-specific modules in its promoter region (Benfey, et al., EMBO J., 8:2195-2202, 1989).

A suitable stem-specific promoter is that described in U.S. Pat. No. 5,625,136 (incorporated herein by reference in its entirety), which drives expression of the maize trpA gene. Plastid specific promoters include the PrbcL promoter (Allison, et al., EMBO J. 15:2802-2809, 1996; Shiina, et al., Plant Cell, 10:1713-1722, 1998); the PpsbA promoter (Agrawal, et al., Nucl. Acids Res. 29:1835-1843, 2001); the Prrn 16 promoter (Svab and Maliga, Proc. Natl. Acad. Sci. USA 90:913-917, 1993, Allison, et al., EMBO J. 15:2802-2809, 1996); and the PaccD promoter (WO 97/06250 (incorporated herein by reference in its entirety); Hajdukiewicz et al., EMBO J. 16:4041-4048, 1997).

Inducible promoters such as chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Inducible promoters are well-known and widely available to those of ordinary skill in the art, which were used successfully in plants (Padidam, Curr. Opin. Plant Biol. 6:169, 2003; Wang, et al., Trans. Res.:12, 529, 2003; Gatz and Lenk, Trends Plant Sci. 3:352, 1998). These inducible systems may be activated by chemicals such as tetracycline, pristamycin, pathogen, light, glucocorticoid, estrogen, copper, herbicide safener, ethanol, IPTG (iso-propyl β-D-1-thiogalactopyranoside), and pathogens. Suitable chemical-inducible promoters include, but are not limited to, the maize In2-2 promoter, which is activated by benzenesulfonamide herbicide safeners; the maize GST promoter, which is activated by hydrophobic electrophilic compounds that are used as pre-emergent herbicides; and the tobacco PR-la promoter, which is activated by salicylic acid. Other chemical-regulated promoters of interest include steroid-responsive promoters (see, e.g., the glucocorticoid-inducible promoter in Schena, et al., Proc. Natl. Acad. Sci. USA, 88:10421-10425, 1991; and McNellis, et al., Plant J., 14:247-257, 1998) and tetracycline-inducible and tetracycline-repressible promoters (see, e.g., Gatz, et al., Mol. Gen. Genet. 227:229-237, 1991, and U.S. Pat. Nos. 5,814,618 and 5,789,156, herein incorporated by reference in their entirety).

Another suitable category of inducible promoters is wound inducible promoters. Numerous promoters have been described which are expressed at wound sites, including those described by Stanford, et al., Mol. Gen. Genet. 215:200-208, 1989; Xu, et al., Plant Mol. Biol. 22:573-588, 1993; Logemann, et al., Plant Cell, 1:151-158, 1989; Rohrmeier and Lehle, Plant Mol. Biol., 22:783-792, 1993; Firek, et al., Plant Mol. Biol., 22:129-142, 1993, and Warner, et at, Plant J. 3:191-201, 1993.

The DNA sequence between the transcription initiation site and the start of the coding sequence, i.e., the untranslated leader sequence, can also influence gene expression. One may thus wish to employ a particular leader sequence with a transformation construct of the disclosure. In certain embodiments, leader sequences are contemplated to include those which comprise sequences predicted to direct optimum expression of the attached gene, i.e., to include a consensus leader sequence which may increase or maintain mRNA stability and prevent inappropriate initiation of translation. The choice of such sequences will be known to those of skill in the art in light of the present disclosure. In some embodiments, sequences that are derived from genes that are highly expressed in plants may be used for expression of OsACBP2 coding sequences.

Numerous sequences have been found to enhance gene expression from within the transcriptional unit and can be used in conjunction with the genes of interest to increase the gene expression in transgenic plants. For example, various intron sequences such as introns of the maize Adh1 gene have been shown to enhance expression, particularly in monocotyledonous cells. In addition, a number of non-translated leader sequences derived from viruses are also known to enhance expression and are particularly effective in dicotyledonous cells.

B. Terminators

Transformation constructs prepared in accordance with the disclosure may include a 3′ end DNA sequence that acts as a signal to terminate transcription and allow for the polyadenylation of the mRNA produced by coding sequences operably linked to a promoter. Non-limiting examples of terminators that may be used in this context include those from the nopaline synthase gene of Agrobacterium tumefaciens (nos 3′ end) (Bevan, et al., Nucl. Acids Res. 11:369-385, 1983), the terminator for the T7 transcript from the octopine synthase gene of Agrobacterium tumefaciens, and the 3′ end of the protease inhibitor I or II gene from potato or tomato. Regulatory elements such as an Adh intron (Callis, et al., Genes Dev. 1:1183-1200, 1987), sucrose synthase intron (Vasil, et al., Plant Physiol. 91:1575-1579, 1989) or TMV omega element (Gallie, et al., The Plant Cell 1:301-311, 1989), may further be included in certain embodiments where desired. Other transcriptional terminators are those known to function in plants and include, but are not limited to, the CaMV 35S terminator, the tm1 terminator, and the pea rbcS E9 terminator, which are used in both monocotyledonous and dicotyledonous plants.

C. Transit Signal or Targeting Peptides

Sequences that are joined to the coding sequence of an expressed gene, which are removed post-translationally from the initial translation product and which facilitate the transport of the protein into or through intracellular or extracellular membranes, are termed transit (usually into vacuoles, vesicles, plastids and other intracellular organelles) and signal sequences (usually to the endoplasmic reticulum, Golgi apparatus and outside of the cellular membrane). By facilitating the transport of the protein into compartments inside and outside the cell, these sequences may increase the accumulation of gene products by protecting them from proteolytic degradation. These sequences also allow for additional mRNA sequences from highly expressed genes to be attached to the coding sequence of the genes. Since mRNA being translated by ribosomes is more stable than naked mRNA, the presence of translatable mRNA in front of the gene may increase the overall stability of the mRNA transcript from the gene and thereby increase synthesis of the gene product. Since transit and signal sequences are usually post-translationally removed from the initial translation product, the use of these sequences allows for the addition of extra translated sequences that may not appear on the final polypeptide. It further is contemplated that targeting of certain proteins may be desirable in order to enhance the stability of the protein (U.S. Pat. No. 5,545,818, incorporated herein by reference in its entirety).

Additionally, vectors may be constructed and employed in the intracellular targeting of a specific gene product within the cells of a transgenic plant or in directing a protein to the extracellular environment. This generally will be achieved by joining a DNA sequence encoding a transit or signal peptide sequence to the coding sequence of a particular gene. The resultant transit or signal peptide will transport the protein to a particular intracellular or extracellular destination, respectively, and will then be post-translationally removed.

The disclosed vectors may further include, within the region that encodes the protein of interest, one or more nucleotide sequences encoding a targeting sequence. A “targeting sequence” is a nucleotide sequence that encodes an amino acid sequence or motif that directs the encoded protein of interest to a particular cellular compartment, resulting in localization or compartmentalization of the protein. Presence of a targeting amino acid sequence in a protein typically results in translocation of all or part of the targeted protein across an organelle membrane and into the organelle interior. Alternatively, the targeting peptide may direct the targeted protein to remain embedded in the organelle membrane. The targeting sequence or region of a targeted protein may contain a string of contiguous amino acids or a group of noncontiguous amino acids. The targeting sequence can be selected to direct the targeted protein to a plant organelle such as a nucleus, a microbody (e.g., a peroxisome, or a specialized version thereof such as a glyoxysome), an endoplasmic reticulum, an endosome, a vacuole, a plasma membrane, a cell wall, mitochondria, a chloroplast or a plastid.

A chloroplast targeting sequence is any peptide sequence that can target a protein to the chloroplasts or plastids, such as the transit peptide of the small subunit of the alfalfa ribulose-biphosphate carboxylase (Khoudi, et al., Gene, 197:343-351, 1997). A peroxisomal targeting sequence refers to any peptide sequence, either N-terminal, internal, or C-terminal, that can target a protein to the peroxisomes, such as the plant C-terminal targeting tripeptide SKL (Banjoko and Trelease, Plant Physiol. 107:1201-1208, 1995). Plastid targeting sequences are known in the art, including the chloroplast small subunit of ribulose-1, 5-bisphosphate carboxylase (Rubisco) (de Castro Silva Filho, et al., Plant Mol. Biol. 30:769-780, 1996; Schnell, et al., J. Biol. Chem. 266:3335-3342, 1991); 5-(enolpyruvyl)shikimate-3-phosphate synthase (EPSPS) (Archer, et al., J. Bioenerg. Biomemb. 22:789-810, 1990); tryptophan synthase (Zhao, et al., J. Biol. Chem. 270:6081-6087, 1995); plastocyanin (Lawrence, et al., J. Biol. Chem. 272:20357-20363, 1997); chorismate synthase (Schmidt, et al., J. Biol. Chem. 268:27447-27457, 1993); and the light harvesting chlorophyll a/b binding protein (LHBP) (Lamppa, et al., J. Biol. Chem. 263:14996-14999, 1988). See also Von Heijne, et al., Plant Mol. Biol. Rep. 9:104-126, 1991; Clark, et al., J. Biol. Chem. 264:17544-17550, 1989; Della-Cioppa, et al., Plant Physiol. 84:965-968, 1987; Romer, et al., Biochem. Biophys. Res. Commun. 196:1414-1421, 1993; and Shah, et al., Science, 233:478-481, 1986. Alternative plastid targeting signals have also been described in U.S. Patent Application Publication No. 2008/0263728 (incorporated herein by reference in its entirety); Miras, et al., J. Biol. Chem. 277:47770-47778, 2002; and Miras, et al., J. Biol. Chem. 282:29482-29492, 2007.

D. Marker Genes

By employing a selectable or screenable marker, one can provide or enhance the ability to identify transformants. “Marker genes” are genes that impart a distinct phenotype to cells expressing the marker protein and thus allow such transformed cells to be distinguished from cells that do not have the marker. Such genes may encode either a selectable or screenable marker, depending on whether the marker confers a trait which one can “select” for by chemical means, i.e., through the use of a selective agent (e.g., a herbicide, antibiotic, or the like), or whether it is simply a trait that one can identify through observation or testing, i.e., by “screening” (e.g., the green fluorescent protein). Of course, many examples of suitable marker proteins are known to the art and can be employed in the practice of the disclosure.

Selectable marker genes that have been used extensively in plants include, but are not limited to, the neomycin phosphotransferase gene nptII (U.S. Pat. Nos. 5,034,322 and 5,530,196, incorporated herein by reference in their entirety), hygromycin resistance gene (U.S. Pat. No. 5,668,298, incorporated herein by reference in its entirety), the bar gene encoding resistance to phosphinothricin (U.S. Pat. No. 5,276,268, incorporated herein by reference in its entirety), the expression of aminoglycoside 3′-adenyltransferase (aadA) to confer spectinomycin resistance (U.S. Pat. No. 5,073,675, incorporated herein by reference in its entirety), the use of inhibition resistant 5-enolpyruvyl-3-phosphoshikimate synthetase (U.S. Pat. No. 4,535,060, incorporated herein by reference in its entirety) and methods for producing glyphosate tolerant plants (U.S. Pat. Nos. 5,463,175 and 7,045,684, incorporated herein by reference in their entirety). Methods of plant selection that do not use antibiotics or herbicides as a selective agent have been previously described and include expression of glucosamine-6-phosphate deaminase to inactive glucosamine in plant selection medium (U.S. Pat. No. 6,444,878, incorporated herein by reference in its entirety), and a positive/negative system that utilizes D-amino acids (Erikson, et al., Nat Biotechnol, 22:455-458, 2004). European Patent Publication No. EP 0 530 129 describes a positive selection system which enables the transformed plants to outgrow the non-transformed lines by expressing a transgene encoding an enzyme that activates an inactive compound added to the growth media. U.S. Pat. No. 5,767,378 (incorporated herein by reference in its entirety) describes the use of mannose or xylose for the positive selection of transgenic plants. Methods for positive selection using sorbitol dehydrogenase to convert sorbitol to fructose for plant growth have also been described (see WO 2010/102293). Screenable marker genes include the β-glucuronidase gene (U.S. Pat. No. 5,268,463, incorporated herein by reference in its entirety) and native or modified green fluorescent protein gene (Cubitt, et al., Trends Biochem. Sci. 20:448-455, 1995; Pan, et al., Plant Physiol., 112:893-900, 1996.

Many selectable marker coding regions are known and could be used with the present disclosure including, but not limited to, neo (Potrykus, et al., Mol. Gen. Genet. 199:183-188, 1985), which provides kanamycin resistance and can be selected for using kanamycin, G418, paromomycin, etc.; a mutant EPSP synthase protein (Hinchee, et al., Bio/Technol. 6:915-922, 1988) conferring glyphosate resistance; a nitrilase such as bxn from Klebsiella ozaenae which confers resistance to bromoxynil (Stalker, et al., Science 242:419-423, 1988); a mutant acetolactate synthase (ALS) which confers resistance to imidazolinone, sulfonylurea or other ALS inhibiting chemicals (European Patent Application 154204, 1985); a methotrexate resistant DHFR (Thillet, et al., J. Biol. Chem. 263:12500-12508, 1988), a dalapon dehalogenase that confers resistance to the herbicide dalapon (Buchanan-Wollaston, et al., Plant Cell Reports 11:627-631, 1992); or a mutated anthranilate synthase that confers resistance to 5-methyl tryptophan (Li and Last, Plant Physiol. 110:51-59, 1996).

An illustrative embodiment of selectable marker capable of being used in systems to select transformants are those that encode the enzyme phosphinothricin acetyltransferase, such as the bar gene from Streptomyces hygroscopicus or the pat gene from Streptomyces viridochromogenes. The enzyme phosphinothricin acetyl transferase (PAT) inactivates the active ingredient in the herbicide bialaphos, phosphinothricin (PPT). PPT inhibits glutamine synthetase (Murakami, et al., Mol. Gen. Genet. 205:42-50, 1986; Twell, et al., Plant Physiol. 91:1270-1274, 1989), causing rapid accumulation of ammonia and cell death.

Transformation events can also be selected through visualization of fluorescent proteins such as the fluorescent proteins from the nonbioluminescent Anthozoa species which include DsRed, and a red fluorescent protein from the Discosoma genus of coral (Matz, et al., Nat Biotechnol, 17:969-973, 1999). An improved version of the DsRed protein has been developed (Bevis and Glick, Nat Biotech, 20:83-87, 2002) for reducing aggregation of the protein. Visual selection can also be performed with the yellow fluorescent proteins (YFP) including the variant with accelerated maturation of the signal (Nagai, et al., Nat Biotech., 20:87-90, 2002), the blue fluorescent protein, the cyan fluorescent protein, and the green fluorescent protein (Sheen, et al., Plant J, 8:777-784, 1995; Davis and Vierstra, Plant Mol. Biol. 36:521-528, 1998). A summary of fluorescent proteins can be found in Tzfira, et al., Plant Mol. Biol. 57:503-516, 2005; and Verkhusha and Lukyanov, Nat Biotech, 22:289-296, 2004. Improved versions of many of the fluorescent proteins have been made for various applications. Use of the improved versions of these proteins or the use of combinations of these proteins for selection of transformants will be obvious to those skilled in the art. It is also practical to simply analyze progeny from transformation events for the presence of the OsACBP2 thereby avoiding the use of any selectable marker.

For plastid transformation constructs, a preferred selectable marker is the spectinomycin-resistant allele of the plastid 16S ribosomal RNA gene (Staub and Maliga, Plant Cell 4:39-45, 1992; Svab, et al., Proc. Natl. Acad. Sci. USA 87:8526-8530, 1990). Selectable markers that have since been successfully used in plastid transformation include the bacterial aadA gene that encodes aminoglycoside 3′-adenyltransferase (AadA) conferring spectinomycin and streptomycin resistance (Svab, et al., Proc. Natl. Acad. Sci. USA 90:913-917, 1993), nptII that encodes aminoglycoside phosphotransferase for selection on kanamycin (Carrer, et al., Mol. Gen. Genet. 241:49-56, 1993; Lutz, et al., Plant J. 37:906-913, 2004; Lutz, et al., Plant Physiol., 145:1201-1210, 2007), aphA6, another aminoglycoside phosphotransferase (Huang, et al., Mol. Genet. Genomics, 268:19-27, 2002), and chloramphenicol acetyltransferase (Li, et al., Plant Mol. Biol. 76:443-451, 2010). Another selection scheme has been reported that uses a chimeric betaine aldehyde dehydrogenase gene (BADH) capable of converting toxic betaine aldehyde to nontoxic glycine betaine (Daniell, et al., Curr. Genet. 39:109-116, 2001).

### Transgenic Plants/Plant Materials

A wide variety of plants and plant cells can be engineered to express an OsACBP2 polypeptide. Plant materials such as leaves, stems, roots, flowers or flower parts, fruits, pollen, egg cells, zygotes, seeds, cuttings, cell or tissue cultures, or any other part or product of a plant can thus be obtained, thus genetically modified and exhibiting improved grain size and/or weight.

The genetically modified plant or plant material comprises one or more genes encoding an OsACBP2 polypeptide or a functional fragment of OsACBP2. In some embodiments the genetically modified plant/plant material comprises two nucleotide sequences encoding two or more OsACBP2, which may be contained on separate expression vectors under the control of separate promoters, or on single expression vector under the control of a common promoter.

In some embodiments, suitable plants and plant cells for engineering include monocotyledonous and dicotyledonous plants, such as grain crops (e.g., wheat, maize, rice, millet, barley), tobacco, fruit crops (e.g., tomato, strawberry, orange, grapefruit, banana), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); oil crops (e.g., sunflower, rape seed); and plants used for experimental purposes (e.g., Arabidopsis). Other examples include plants that are typically grown in groups of more than 10 in order to harvest the entire plant or a part of the plant, e.g., a fruit, a crop, a tree (e.g., fruit trees, trees grown for wood production, trees grown for decoration, etc.), a flower of any kind (e.g., plants grown for purposes of decoration following their harvest), and cactuses. Further examples of suitable plants engineered to express OsACBP2 include Viridiplantae, Streptophyta, Embryophyta, Tracheophyta, Euphyllophytes, Spermatophyta, Magnoliophyta, Liliopsida, Commelinidae, Poales, Poaceae, Oryza, Oryza sativa, Zea, Zea mays, Hordeum, Hordeum vulgare, Triticum, Triticum aestivum, Eudicotyledons, Core eudicots, Asteridae, Euasterids, Rosidae, Eurosids II, Brassicales, Brassicaceae, Arabidopsis, Magnoliopsida, Solananae, Solanales, Solanaceae, Solanum, and Nicotiana. Additional plants that can be transformed using the vectors described herein include, but are not limited to, species from the genera Anacardium, Arachis, Asparagus, Atropa, Avena, Brassica, Citrus, Citrullus, Capsicum, Carthamus, Cocos, Coffea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Manihot, Majorana, Medicago, Nicotiana, Olea, Oryza, Panieum, Panneserum, Persea, Phaseolus, Pistachia, Pisum, Pyrus, Prunus, Raphanus, Ricinus, Secale, Senecio, Sinapis, Solanum, Sorghum, Theobromus, Trigonella, Titicum, Vicia, Vitis, Vigna, and Zea.

### Genetic Transformation

Additionally provided herein are transgenic plants transformed with the above-identified recombinant vectors encoding OsACBP2, or a sequence modulating up-regulation thereof. Transgenic plants and plant cells/plant materials may be obtained by engineering one or more of the vectors expressing an OsACBP2 polypeptide or a functional fragment of OsACBP2 as described herein into a variety of plant cell types, including but not limited to, protoplasts, tissue culture cells, tissue and organ explants, pollens, embryos, as well as whole plants. Transformation methods as well as methods for introducing nucleotide sequences into plants may vary depending on the type of plant or plant cell targeted for transformation.

Suitable methods for transformation of plant or other cells for use with the current disclosure are believed to include virtually any method by which DNA can be introduced into a cell, such as by direct delivery of DNA such as by PEG-mediated transformation of protoplasts (Omirulleh, et al., Plant Mol. Biol. 21:415-428, 1993), microinjection (Crossway, et al., Biotechniques 4:320-334, 1986), by desiccation/inhibition-mediated DNA uptake (Potrykus, et al., Mol. Gen. Genet. 199:183-188, 1985), by electroporation (U.S. Pat. No. 5,384,253, specifically incorporated herein by reference in its entirety), by agitation with silicon carbide fibers (Kaeppler, et al., Plant Cell Reports 9:415-418, 1990; U.S. Pat. Nos. 5,302,523 and 5,464,765, both specifically incorporated herein by reference in its entirety), by Agrobacterium-mediated transformation (U.S. Pat. Nos. 5,591,616 and 5,563,055; both specifically incorporated herein by reference) and by acceleration of DNA coated particles (U.S. Pat. Nos. 5,550,318; 5,538,877; and 5,538,880; each specifically incorporated herein by reference in its entirety). Also see Weissinger, et al., Ann. Rev. Genet. 22:421-477, 1988; Sanford, et al., Part. Sci. Tech. 5:27-37, 1987 (onion); Christou, et al., Plant Physiol. 87:671-674, 1988 (soybean); McCabe, et al., BioTechnology 6:923-926, 1988 (soybean); Finer and McMullen, In Vitro Cell Dev. Biol., 27P:175-182, 1991 (soybean); Singh, et al., Theor. Appl. Genet. 96:319-324, 1998 (soybean); Dafta, et al., Biotechnology 8:736-740, 1990 (rice); U.S. Pat. Nos. 5,240,855, 5,322,783 and 5,324,646 (incorporated herein by reference in their entirety) (maize); U.S. Pat. No. 5,736,369 (incorporated herein by reference in its entirety) (cereals); Bytebier, et al., Proc. Natl. Acad. Sci. USA 84:5345-5349, 1987 (Liliaceae); De Wet, et al., in The Experimental Manipulation of Ovule Tissues, ed. Chapman, et al. (Longman, N.Y.), pp. 197-209 (1985) (pollen); Kaeppler, et al., Plant Cell Reports 9:415-418, 1990 and Kaeppler, et al., Theor. Appl. Genet. 84:560-566, 1992 (whisker-mediated transformation); D'Halluin, et al., Plant Cell 4:1495-1505, 1992 (electroporation); Li, et al., Plant Cell Reports 12:250-255, 1993; Christou and Ford, Annals of Botany 75:407-413, 1995 (rice); Osjoda, et al., Nat. Biotechnol. 14:745-750, 1996 (maize via Agrobacterium tumefaciens). Through the application of techniques such as these, the cells of virtually any plant species may be stably transformed, and these cells developed into transgenic plants.

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well-known in the art. See, for example, the methods described by Horsch, et al. (Science 227:1229-1231, 1985), Rogers and Klee (Plant DNA Infectious Agents, Chapter 7, Springer-Verlag/Wein, 1987) and U.S. Pat. No. 5,563,055, specifically incorporated herein by reference in its entirety.

Agrobacterium-mediated transformation is most efficient in dicotyledonous plants and is the preferable method for transformation of dicots, including Arabidopsis, tobacco, tomato, alfalfa and potato. Indeed, while Agrobacterium-mediated transformation has been routinely used with dicotyledonous plants for a number of years, including alfalfa (Thomas, et al., Plant Sci. 69:189-198, 1990), it has only more recently become applicable to monocotyledonous plants. Advances in Agrobacterium-mediated transformation techniques have now made the technique applicable to nearly all monocotyledonous plants. For example, Agrobacterium-mediated transformation techniques have now been applied to rice (Hiei, et al., Plant Mol. Biol. 35:205-218, 1997; U.S. Pat. No. 5,591,616, specifically incorporated herein by reference in its entirety), wheat (McCormac, et al., Euphytica 99:17-25, 1998), barley (Tingay, et al., The Plant Journal 11:1369-1376, 1997) and maize (Ishidia, et al., Nature Biotechnology 14:745-750, 1996).

One also may employ protoplasts for electroporation transformation of plants (Bates, Mol. Biotechnol. 2:135-145, 1994; Lazzeri, Methods Mol. Biol. 49:95-106, 1995). Another method for delivering transforming DNA segments to plant cells in accordance with the disclosure is microprojectile bombardment (U.S. Pat. Nos. 5,550,318; 5,538,880; 5,610,042; and PCT Application WO 94/09699; each of which is specifically incorporated herein by reference in its entirety). In this method, particles may be coated with nucleic acids and delivered into cells by a propelling force. Methods for protoplast transformation and/or gene gun for Agrisoma technology are described in WO 2010/037209. Methods for transforming plant protoplasts are available including transformation using polyethylene glycol (PEG), electroporation, and calcium phosphate precipitation (see e.g., Potrykus, et al., Mol. Gen. Genet. 199:183-188, 1985; Potrykus, et al., Plant Mol. Biol. Rep. 3:117-128, 1985. Methods for plant regeneration from protoplasts have also been described (Evans, et al., in Handbook of Plant Cell Culture, Vol 1, (Macmillan Publishing Co., New York, 1983); Vasil, in Cell Culture and Somatic Cell Genetics (Academic, Orlando, 1984)).

Methods for transformation of plastids such as chloroplasts are known in the art. See, for example, Svab, et al., Proc. Natl. Acad. Sci. USA 87:8526-8530, 1990; Svab and Maliga, Proc. Natl. Acad. Sci. USA 90:913-917, 1993; Svab and Maliga, EMBO J. 12:601-606, 1993 and Staub and Maliga, Plant J. 6:547-553, 1994; Kuehnle, U.S. Patent Application Publication No. 2009/7618819. The method relies on particle gun delivery of DNA containing a selectable marker and targeting of the DNA to the plastid genome through homologous recombination. Additionally, plastid transformation may be accomplished by transactivation of a silent plastid-borne transgene by tissue-preferred expression of a nuclear-encoded and plastid-directed RNA polymerase (McBride, et al., Proc. Natl. Acad. Sci. USA 91:7301-7305, 1994) or by use of an integrase, such as the phiC31 phage site-specific integrase, to target the gene insertion to a previously inserted phage attachment site (Lutz, et al., Plant J 37:906-913, 2004). Plastid transformation vectors can be designed such that the transgenes are expressed from a promoter sequence that has been inserted with the transgene during the plastid transformation process, or alternatively, from an endogenous plastidial promoter such that an extension of an existing plastidial operon is achieved (Herz, et al., Transgenic Res. 14:969-982, 2005). An alternative method for plastid transformation as described in WO 2010/061186, wherein RNA produced in the nucleus of a plant cell can be targeted to the plastid genome, can also be used. Inducible gene expression from the plastid genome using a synthetic riboswitch has also been reported (Verhounig, et al., Proc. Natl. Acad. Sci. USA 107:6204-6209, 2010). Methods for designing plastid transformation vectors are described by Lutz, et al., Plant Physiol, 145:1201-1210, 2007.

The transgenic plants of the present disclosure expressing heterologous OsACBP2 and exhibiting increased grain size and weight can be of any species. The plants can be an R0 transgenic plant (i.e., a plant derived from the original transformed tissue). The plants can also be a progeny plant of any generation of an R0 transgenic plant, wherein the transgenic plant comprises the nucleic acid sequence from the R0 transgenic plant.

Seeds of the above-described transgenic plants may also be provided, particularly where the seed comprises the nucleic acid sequence. Additionally contemplated are host cells transformed with the above-identified recombinant vector. In some embodiments, the host cell is a plant cell.

The plants of these embodiments having increased or enhanced expression of OsACBP2 and exhibiting increased grain size and weight may be of any species. The species may be any monocotyledonous or dicotyledonous plant, such as those described herein. One of skill in the art will recognize that the present disclosure may be applied to plants of other species by employing methods described herein and others known in the art.

Tissue cultures may be used in certain transformation techniques for the preparation of cells for transformation and for the regeneration of plants therefrom. Maintenance of tissue cultures requires use of media and controlled environments. “Media” refers to the numerous nutrient mixtures that are used to grow cells in vitro, that is, outside of the intact living organism. A medium usually is a suspension of various categories of ingredients (salts, amino acids, growth regulators, sugars, buffers) that are required for growth of most cell types. However, each specific cell type requires a specific range of ingredient proportions for growth, and an even more specific range of formulas for optimum growth. The rate of cell growth also will vary among cultures initiated with the array of media that permit growth of that cell type.

### Production and Characterization of Stably Transformed Plants

After effecting delivery of exogenous DNA to recipient cells, the next steps generally concern identifying the transformed cells for further culturing and plant regeneration. In order to improve the ability to identify transformants, one may desire to employ a selectable or screenable marker gene with a transformation vector prepared in accordance with the present disclosure. In this case, one would then generally assay the potentially transformed cell population by exposing the cells to a selective agent or agents, or one would screen the cells for the desired marker gene trait. Potentially transformed cells then are exposed to the selective agent. In the population of surviving cells will be those cells where, generally, the resistance-conferring gene has been integrated and expressed at sufficient levels to permit cell survival. Cells may be tested further to confirm stable integration of the exogenous DNA. Following transformation by any one of the methods described above, procedures that can be used to obtain a transformed plant expressing the transgenes include, but are not limited to: selecting the plant cells that have been transformed on a selective medium; regenerating the plant cells that have been transformed to produce differentiated plants; selecting transformed plants expressing the transgene producing the desired level of desired polypeptide(s) in the desired tissue and cellular location.

A transformed plant cell, callus, tissue, or plant may be identified and isolated by selecting or screening the engineered plant material for traits encoded by the selection marker genes present on the introduced expression cassette. For instance, selection may be performed by growing the engineered plant material on media containing inhibitory amount of the antibiotic or herbicide to which the transforming gene construct confers resistance. Particularly, the selectable marker gene nptII, which specifies kanamycin-resistance, can be used in nuclear transformation. As another example, transformed plants and plant materials may be identified by screening for the activities of any visible marker genes (e.g., the β-glucuronidase, luciferase, B or C1 genes) that may be present on the vectors described herein. Such selection and screening methodologies are well-known to those skilled in the art. Alternatively or in addition, the transformed plant cell, callus, tissue, or plant screening may be screened for improved grain size and/or weight as taught herein.

One herbicide which constitutes a desirable selection agent is the broad-spectrum herbicide bialaphos. Another example of a herbicide which is useful for selection of transformed cell lines in the practice of the disclosure is the broad-spectrum herbicide glyphosate. Glyphosate inhibits the action of the enzyme EPSPS which is active in the aromatic amino acid biosynthetic pathway. Inhibition of this enzyme leads to starvation for the amino acids phenylalanine, tyrosine, and tryptophan and secondary metabolites derived therefrom. U.S. Pat. No. 4,535,060 describes the isolation of EPSPS mutations which confer glyphosate resistance on the EPSPS of Salmonella typhimurium, encoded by the gene aroA. The EPSPS gene from Zea mays was cloned and mutations similar to those found in a glyphosate resistant aroA gene were introduced in vitro. Mutant genes encoding glyphosate resistant EPSPS enzymes are described in, for example, International Patent Application Publication Number WO 97/4103.

The transformed cells, identified by selection or screening and cultured in an appropriate medium that supports regeneration, will then be allowed to mature into plants. Developing plantlets can be transferred to soilless plant growth mix, and hardened, e.g., in an environmentally controlled chamber, for example, at about 85% relative humidity, 600 ppm CO₂, and 25-250 microeinsteins m-2 s-1 of light. Plants may be matured in a growth chamber or greenhouse. Plants can be regenerated after a transformant is identified, depending on the initial tissue. During regeneration, cells are grown on solid media in tissue culture vessels. Illustrative embodiments of such vessels are petri dishes and Plant Cons. Regenerating plants can be grown at about 19 to 28° C., for example. After the regenerating plants have reached the stage of shoot and root development, they may be transferred to a greenhouse for further growth and testing.

To confirm the presence of the exogenous DNA or “transgene(s)” in the regenerating plants, a variety of assays may be performed. Physical, biochemical or “molecular biological” methods may be used to identify plant or plant cell transformants containing the gene constructs/vectors described herein. These methods include, but are not limited to: 1) Southern analysis or PCR amplification for detecting and determining the structure of the recombinant DNA insert; 2) Northern blot, S1 RNase protection, primer-extension or reverse transcriptase-PCR amplification for detecting and examining RNA transcripts of the gene constructs; 3) enzymatic assays for detecting enzyme activity, where such gene products are encoded by the gene construct; 4) protein gel electrophoresis (PAGE), Western blot techniques, immunoprecipitation, or enzyme-linked immunoassays, where the gene construct products are proteins. Additional techniques, such as in situ hybridization, enzyme staining, and immunostaining, may also be used to detect the presence or expression of the recombinant construct in specific plant organs and tissues. The aforementioned methods/assays are well-known to those skilled in the art. Such assays include, for example, detecting the presence of a protein product, e.g., by immunological means (ELISAs and Western blots) or by enzymatic function; plant part assays, such as leaf or root assays; and also, by analyzing the phenotype of the whole regenerated plant.

The expression of a gene product is often determined by evaluating the phenotypic results of its expression. These assays also may take many forms including but not limited to analyzing changes in the chemical composition, morphology, or physiological properties of the plant. Chemical composition may be altered by expression of genes encoding enzymes or storage proteins which change amino acid composition and may be detected by amino acid analysis, or by enzymes that change starch quantity which may be analyzed by near infrared reflectance spectrometry. Morphological changes may include greater stature or thicker stalks. Most often changes in response of plants or plant parts to imposed treatments are evaluated under carefully controlled conditions termed bioassays. Such assays for grain size and weight are well-described herein.

The cells that have been transformed may be grown into plants in accordance with conventional techniques. See, e.g., McCormick, et al., Plant Cell Reports 5:81-84, 1986. These plants may be grown, pollinated with either the same transformed variety or different varieties, to result in hybrids having constitutive expression of the desired phenotypic characteristic identified. Two or more generations may be grown to ensure that constitutive expression of the desired phenotypic characteristic is stably maintained and inherited, and then seeds are harvested to ensure constitutive expression of the desired phenotypic characteristic. An isolated transformant may be regenerated into a plant and progeny thereof (including the immediate and subsequent generations) via sexual or asexual reproduction or growth. Alternatively, the engineered plant material may be regenerated into a plant before subjecting the derived plant to selection or screening for the marker gene traits. Procedures for regenerating plants from plant cells, tissues or organs, either before or after selecting or screening for marker gene(s), are well-known to those skilled in the art.

In plastid transformation procedures, further rounds of regeneration of plants from explants of a transformed plant or tissue can be performed to increase the number of transgenic plastids such that the transformed plant reaches a state of homoplasmy (all plastids contain uniform plastomes containing transgene insert).

### Breeding Plants of the Invention

In addition to direct transformation of a particular plant genotype with a construct prepared according to the current disclosure, transgenic plants may be made by crossing a plant having a selected DNA of the disclosure to a second plant lacking the construct. For example, a selected OsACBP2 coding sequence can be introduced into a particular plant variety by crossing, without the need for ever directly transforming a plant of that given variety. Therefore, the current disclosure not only encompasses a plant directly transformed or regenerated from cells that have been transformed in accordance with the current disclosure, but also the progeny of such plants. As used herein, the term “progeny” denotes the offspring of any generation of a parent plant prepared in accordance with the instant disclosure, wherein the progeny comprises a selected DNA construct prepared in accordance with the disclosure. “Crossing” a plant to provide a plant line having one or more added transgenes relative to a starting plant line, as disclosed herein, is defined as the techniques that result in a transgene of the disclosure being introduced into a plant line by crossing a plant of a starting line with a plant of a donor plant line that comprises a transgene of the disclosure. To achieve this one could, for example, perform the following steps: (a) plant seeds of the first (starting line) and second (donor plant line that comprises a transgene of the disclosure) parent plants; (b) grow the seeds of the first and second parent plants into plants that bear flowers; (c) pollinate a flower from the first parent plant with pollen from the second parent plant; and (d) harvest seeds produced on the parent plant bearing the fertilized flower. Backcrossing is herein defined as the process including the steps of: (a) crossing a plant of a first genotype containing a desired gene, DNA sequence or element to a plant of a second genotype lacking the desired gene, DNA sequence or element; (b) selecting one or more progeny plant containing the desired gene, DNA sequence or element; (c) crossing the progeny plant to a plant of the second genotype; and (d) repeating steps (b) and (c) for the purpose of transferring a desired DNA sequence from a plant of a first genotype to a plant of a second genotype. In any one or more generations of crossing, selection may be made for increased grain size and/or weight, yielding progeny with increased grain size and/or weight.

Introgression of a DNA element into a plant genotype is defined as the result of the process of backcross conversion. A plant genotype into which a DNA sequence has been introgressed may be referred to as a backcross converted genotype, line, inbred, or hybrid. Similarly a plant genotype lacking the desired DNA sequence may be referred to as an unconverted genotype, line, inbred, or hybrid.

Methods for Identifying Genes that Improve Grain Size and/or Weight

Methods are provided for identifying variants and homologs of OsACBP2 that promote grain size and/or weight. An exemplary screening method involves introducing an exogenous nucleic acid into a host cell, producing a test cell, where the host cell is one that exhibits enhanced grain size and/or weight phenotype and reproduction over the wild-type. When an exogenous nucleic acid comprising a nucleotide sequence that encodes an OsACBP2 polypeptide is introduced into the host cell, grain size of the test transgenic rice plant is enhanced. Thus, an increase in grain size and/or weight indicates that the exogenous nucleic acid encodes an OsACBP2, wherein the encoded polypeptide is produced at a level and/or has an activity that promotes grain size. The increase in grain size and/or weight is observed to be at least about 10%, at least about 20%, at least about 30%, at least about 40%, at least about 50%, at least about 60%, at least about 70%, at least about 80%, at least about 90%, or more, as compared to a non-genetically-modified host.

To generate a genetically modified host cell exhibiting enhanced grain size, one or more nucleic acids including nucleotide sequences encoding one or more OsACBP2 polypeptides that could promote grain size is introduced stably or transiently into a parent host cell, using established techniques, including, but not limited to, electroporation, calcium phosphate precipitation, DEAE-dextran mediated transfection, liposome-mediated transfection, particle bombardment, Agrobacterium-mediated transformation, and the like. For stable transformation, a nucleic acid will generally further include a selectable marker, e.g., neomycin resistance, ampicillin resistance, tetracycline resistance, chloramphenicol resistance, kanamycin resistance, or a hygromycin resistance marker.

The exogenous nucleic acid is inserted into an expression vector. Expression vectors that are suitable for use in prokaryotic and eukaryotic host cells are known in the art, including, but not limited to, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector suitable to maintain, propagate or express polynucleotides to produce a polypeptide in a host may be used.

The appropriate nucleotide sequence may be inserted into an expression system by those well-known and routine techniques, e.g., those set forth in Sambrook, et al., Molecular Cloning, A Laboratory Manual (2nd Ed., Cold Spring Harbor Laboratory Press, Cold Spring Harbor, N.Y. (1989)). Where a parent host cell has been genetically modified to produce two or more OsACBP2s, nucleotide sequences encoding two or more OsACBP2s, in some embodiments, are each contained on separate expression vectors; or in other embodiments, are contained on a single expression vector, operably linked to a common control element (e.g., a promoter).

In some embodiments, the screening method includes further characterizing a candidate gene product. In these embodiments, the exogenous nucleic acid comprising nucleotide sequence(s) encoding an OsACBP2(s) are isolated from a test cell as described above. The isolated nucleic acid may be subjected to nucleotide sequence analysis, and the amino acid sequence of the gene product deduced from the nucleotide sequence may further be analyzed as well. In some embodiments, the amino acid sequence of the gene product is compared with other amino acid sequences in a public database of amino acid sequences, to determine whether any significant amino acid sequence identity to an amino acid sequence of a known protein exists.

A. Exogenous Nucleic Acids

Exogenous nucleic acids that are suitable for introducing into a host cell, to produce a test cell, include, but are not limited to, naturally-occurring nucleic acids isolated from a cell. Exogenous nucleic acids to be introduced into a host cell may be identified by hybridization under stringent conditions to a nucleic acid encoding OsACBP2.

Naturally-occurring nucleic acids that have been modified (for example, by mutation) before or subsequent to isolation from a cell; synthetic nucleic acids, e.g., nucleic acids synthesized in a laboratory using standard methods of chemical synthesis of nucleic acids, or generated by recombinant methods; synthetic or naturally-occurring nucleic acids that have been amplified in vitro, either within a cell or in a cell-free system; and the like. Exemplary exogenous nucleic acids include, but are not limited to, genomic DNA; RNA; a complementary DNA (cDNA) copy of mRNA isolated from a cell; recombinant DNA; and DNA synthesized in vitro, e.g., using standard cell-free in vitro methods for DNA synthesis. In some embodiments, exogenous nucleic acids are a cDNA library made from cells, either prokaryotic cells or eukaryotic cells. In some embodiments, exogenous nucleic acids are a genomic DNA library made from cells, either prokaryotic cells or eukaryotic cells.

Manual chemical synthesis of DNA may be accomplished using well-established procedures, or automated chemical synthesis can be performed using one of a number of commercially available machines. The nucleotide sequence of the nucleic acids can be modified for optimal expression based on optimization of nucleotide sequence to reflect the codon bias of the host cell. The skilled artisan appreciates the likelihood of successful expression if codon usage is biased towards those codons favored by the host. Determination of preferred codons can be based on a survey of genes derived from the host cell where sequence information is available. Fragments of full-length proteins can be produced by techniques well-known in the art, such as by creating synthetic nucleic acids encoding the desired portions; or by use of Bal 31 exonuclease to generate fragments of a longer nucleic acid.

In still other embodiments, a variant OsACBP2 is encoded by a nucleic acid that hybridizes under stringent conditions to a nucleic acid encoding a OsACBP2 or another OsACBP2 known in the art.

### Definitions

OsACBP2: Polynucleotides or polypeptides of Oryza sativa acyl-CoA-binding protein 2 that can convey improved grain size to the host in which they are overexpressed.

OsACBP2-OEs: Transgenic Oryza sativa overexpressing an OsACBP2 polypeptide.

Chemically Synthesized: The component nucleotides of a sequence of DNA are assembled in vitro.

Construct: A recombinant nucleic acid, generally recombinant DNA, which has been generated for the purpose of expressing specific nucleotide sequence(s), or is to be used in the construction of other recombinant nucleotide sequences.

Control Plant: A plant that does not contain an introduced coding sequence that is being tested, for example an empty vector-transformed (for example, pCXSN) plant, wherein an OsACBP2 polypeptide is not overexpressed.

Cotyledon: The embryonic first leaves of a seedling.

DNA Regulatory Sequences, Control Elements, and Regulatory Elements: Terms that are used interchangeably and refer to transcriptional and translational control sequences, such as promoters, enhancers, polyadenylation signals, terminators, protein degradation signals, and the like, that provide for and/or regulate expression of a coding sequence and/or production of an encoded polypeptide in a host cell.

Endogenous Nucleic Acid: A nucleic acid that is normally found in and/or produced by a given bacterium, organism, or cell in nature. An “endogenous nucleic acid” is also referred to as a “native nucleic acid” or a nucleic acid that is “native” to a given bacterium, organism, or cell.

Exogenous Nucleic Acid: A nucleic acid that is not normally or naturally found in and/or produced by a given bacterium, organism, or cell in nature.

Expression: The combination of intracellular processes, including transcription and translation, undergone by a coding DNA molecule such as a structural gene to produce a polypeptide.

Gene Product: RNA encoded by DNA (or vice versa) or protein that is encoded by an RNA or DNA, where a gene will typically comprise one or more nucleotide sequences that encode a protein, and may also include introns and other non-coding nucleotide sequences.

Genetic Transformation: A process of introducing a DNA sequence or construct (e.g., a vector or expression cassette) into a cell or protoplast in which that exogenous DNA is incorporated into a chromosome or is capable of autonomous replication.

Heterologous: A sequence which is not normally present in a given host genome in the genetic context in which the sequence is currently found. In this respect, the sequence may be native to the host genome, but be rearranged with respect to other genetic sequences within the host sequence. For example, a regulatory sequence may be heterologous in that it is linked to a different coding sequence relative to the native regulatory sequence.

Heterologous Nucleic Acid: A nucleic acid wherein at least one of the following is true: (a) the nucleic acid is foreign (“exogenous,” i.e., not naturally found in) a given host microorganism or host cell; (b) the nucleic acid comprises a nucleotide sequence that is naturally found in (i.e., “endogenous to”) a given host microorganism or host cell but produced in an unnatural amount in the cell (e.g., greater than expected or greater than naturally found); (c) the nucleic acid comprises a nucleotide sequence that differs from the endogenous nucleotide sequence but encodes the same protein (i.e., having the same or substantially the same amino acid sequence) and produced in an unnatural amount in a host cell; (d) the nucleic acid comprises two or more nucleotide sequences that are not found in the same relationship in nature, e.g., the nucleic acid is recombinant. An example of a heterologous nucleic acid is a nucleotide sequence encoding an OsACBP2 operably linked to a transcriptional control element (e.g., a promoter) to which an endogenous OsACBP2 coding sequence is not normally operably linked. Another example of a heterologous nucleic acid is a high copy number plasmid comprising a nucleotide sequence encoding an OsACBP2. Still another example of a heterologous nucleic acid is a nucleic acid encoding an OsACBP2, where a host cell that does not normally produce OsACBP2 is genetically modified with the nucleic acid encoding OsACBP2. In this case, because OsACBP2-encoding nucleic acids are not naturally found in the host cell, the nucleic acid is heterologous to the genetically modified host cell.

Host Cell: An in vivo or in vitro eukaryotic cell, a prokaryotic cell, or a cell from a multicellular organism (e.g., a cell line) cultured as a unicellular entity, which eukaryotic or prokaryotic cells can be, or have been, used as recipients for a nucleic acid (e.g., an expression vector that comprises a nucleotide sequence encoding one or more gene products such as OsACBPs). It is intended to include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. As used herein, the term “a recombinant host cell” or “a genetically modified host cell” refers to a host cell into which a heterologous nucleic acid has been introduced, e.g., an expression vector.

Isolated: Describes a polynucleotide, a polypeptide, or a cell, that is in an environment different from that in which the polynucleotide, the polypeptide, or the cell naturally occurs. An isolated genetically modified host cell may be present in a mixed population of genetically modified host cells.

Modified Plant or Plant Parts: A plant or plant part, whether it is attached or detached from the whole plant. It also includes progeny of the modified plant or plant parts that are produced through sexual or asexual reproduction.

Naturally-Occurring or Native: As applied to a nucleic acid, a cell, or an organism, refers to a nucleic acid, cell, or organism that is found in nature. For example, a polypeptide or polynucleotide sequence that can be isolated from a source in nature and has not been intentionally modified by human in the laboratory is naturally occurring; or, “wild-type” plants are naturally occurring.

Obtaining: When used in conjunction with a transgenic plant cell or transgenic plant, obtaining means either transforming a non-transgenic plant cell or plant to create the transgenic plant cell or plant, or planting transgenic plant seed to produce the transgenic plant cell or plant. Such a transgenic plant seed may be from an R0 transgenic plant or may be from a progeny of any generation thereof that inherits a given transgenic sequence from a starting transgenic parent plant.

Operably Linked: A juxtaposition wherein the components are in a relationship permitting them to function in their intended manner. For example, a promoter is operably linked to a coding sequence if the promoter affects its transcription or expression.

Operon or Single Transcription Unit: Two or more contiguous coding regions that are coordinately regulated by the same one or more controlling elements (e.g., a promoter).

Overexpression: The increase in the expression of a DNA or RNA transcript and/or the function or activity of a protein relative to a control or naturally-occurring counterpart.

Peptide, Polypeptide or Protein: A polymeric form of amino acids of any length, which can include coded and non-coded amino acids, chemically or biochemically modified or derivatized amino acids, and/or polypeptides having modified peptide backbones.

Percent of Sequence Identity: In reference to a polypeptide or polynucleotide to another polynucleotide or polypeptide, means that, when aligned, that percentage of bases or amino acids are the same, and in the same relative position, when comparing the two sequences.

Plant Cell Culture: Cultures of plant units such as protoplasts, cell culture cells, cells in plant tissues, pollen, pollen tubes, ovules, embryo sacs, zygotes and embryos at various stages of development.

Plant Material: Leaves, stems, roots, flowers or flower parts, fruits, pollen, egg cells, zygotes, seeds, cuttings, cell or tissue cultures, or any other part or product of a plant.

Plant Product: Other than a seed or a fruit or vegetable, is intended as a commodity or other products that moves through commerce and is derived from a transgenic plant or transgenic plant part, in which the commodity or other products can be tracked through commerce by detecting nucleotide segments, RNA or proteins that encode or comprise distinguishing portions of the proteins of the present disclosure and are produced in or maintained in the plant or plant tissue or part from which the commodity or other product has been obtained. Such commodity or other products of commerce include, but are not limited to, plant parts, biomass, oil, meal, sugar, animal feed, flour, flakes, bran, lint, and processed seed. Plant parts include, but are not limited to, a plant seed, boll, leaf, flower, stem, pollen, or root. In certain embodiments, the plant part is a non-regenerable portion of said seed, boll, leaf, flower, stem, pollen, or root.

Plant Tissue: A group of plant cells organized into a structural and functional unit. It is intended to include any tissue of a plant, whether in a plant or in culture. It includes, but not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

Polynucleotide or Nucleic Acid: A polymeric form of nucleotides of any length, either ribonucleotides or deoxyribonucleotides. It includes, but not limited to, single-, double-, or multi-stranded DNA or RNA, genomic DNA, cDNA, DNA-RNA hybrids, or a polymer comprising purine and pyrimidine bases or other natural, chemically or biochemically modified, non-natural or derivatized nucleotide bases.

Progeny: The immediate and all subsequent generations of offspring traceable to a parent.

Promoter: A recognition site on a DNA sequence or group of DNA sequences that provides an expression control element for a structural gene and to which RNA polymerase specifically binds and initiates RNA synthesis (transcription) of that gene.

R0 transgenic plant: A plant that has been genetically transformed or has been regenerated from a plant cell or cells that have been genetically transformed.

Recombinant: Refers to a particular nucleic acid (DNA or RNA) that is the product of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non-translated sequences, or introns, which are typically present in eukaryotic genes. Genomic DNA comprising the relevant sequences can also be used in the formation of a recombinant gene or transcriptional unit. Sequences of non-translated DNA may be present at 5′ or 3′ from the open reading frame, where such sequences do not interfere with manipulation or expression of the coding regions, and may indeed act to modulate production of a desired product by various mechanisms (see “DNA regulatory sequences”, above). Thus, as used herein, the term “recombinant” polynucleotide or nucleic acid refers to one which is not naturally occurring (e.g., made by artificial combination of two otherwise separated segments of sequence through human intervention). This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions.

Regeneration: The process of growing a plant from a plant cell (e.g., plant protoplast, callus or explant).

Selected DNA: A DNA segment which one desires to introduce or has introduced into a plant genome by genetic transformation.

Synthetic Nucleic Acids: A nucleic acid that can be assembled from oligonucleotide building blocks that are chemically synthesized using procedures known to those skilled in the art. These building blocks are ligated and annealed to form gene segments that are then enzymatically assembled to construct the entire gene.

Transformation construct: A chimeric DNA molecule which is designed for introduction into a host genome by genetic transformation. Preferred transformation constructs will comprise all of the genetic elements necessary to direct the expression of one or more exogenous genes. In particular embodiments of the instant disclosure, it may be desirable to introduce a transformation construct into a host cell in the form of an expression cassette.

Transformation or Transformed: Used interchangeably herein with the term “genetic modification” or “genetically modified” and refer to a permanent or transient genetic change induced in a cell following introduction of new nucleic acid (i.e., DNA exogenous to the cell). Genetic change (“modification”) can be accomplished either by incorporation of the new DNA into the genome of the host cell, or by transient or stable maintenance of the new DNA as an episomal element. Where the cell is a eukaryotic cell, a permanent genetic change is generally achieved by introduction of the DNA into the genome of the cell or into a plastome of the cell. In prokaryotic cells, permanent changes can be introduced into the chromosome or via extrachromosomal elements such as plasmids, plastids, and expression vectors, which may contain one or more selectable markers to aid in their maintenance in the recombinant host cell.

Transformation Vectors or Expression Cassettes: A nucleic acid sequence encoding an OsACBP2 polypeptide. The vector or expression cassette can optionally comprise a plant expressible promoter, operably linked to the coding sequence, and a terminator, and/or other regulatory elements. In other embodiments, the vector can be designed to introduce the heterologous polypeptide so that it will be expressed under the control of a plant's own endogenous promoter. The plant transformation vectors preferably include a transcriptional initiation, control region(s) and/or termination region. Transcriptional control regions include those that provide for over-expression of the protein of interest in the genetically modified host cell, and/or those that provide for inducible expression, such that when an inducing agent is added to the culture medium, transcription of the coding region of the protein of interest is induced or increased to a higher level than prior to induction.

Transformed cell: A cell in which the DNA complement has been altered by the introduction of an exogenous DNA molecule into that cell.

Transgene: A segment of DNA which has been incorporated into a host genome or is capable of autonomous replication in a host cell and is capable of causing the expression of one or more coding sequences. Exemplary transgenes will provide the host cell, or plants regenerated therefrom, with a novel phenotype relative to the corresponding non-transformed cell or plant. Transgenes may be directly introduced into a plant by genetic transformation, or may be inherited from a plant of any previous generation which was transformed with the DNA segment.

Transgenic plant: A plant or progeny plant of any subsequent generation derived therefrom, wherein the DNA of the plant or progeny thereof contains an introduced exogenous DNA segment not naturally present in a non-transgenic plant of the same strain. The transgenic plant may additionally contain sequences which are native to the plant being transformed, but wherein the “exogenous” gene has been altered in order to alter the level or pattern of expression of the gene, for example, by use of one or more heterologous regulatory or other elements.

Up-regulation: The increase in the expression of a DNA or RNA transcript and/or the function or activity of a protein relative to a control or naturally-occurring counterpart.

Vector: A DNA molecule designed for transformation into a host cell. Some vectors may be capable of replication in a host cell. A plasmid is an exemplary vector, as are expression cassettes obtained therefrom.

### EXAMPLES

The following examples are included to demonstrate illustrative embodiments of the disclosure. It should be appreciated by those of skill in the art that the techniques disclosed in the examples which follow represent techniques discovered by the inventors to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

Unless indicated otherwise, parts are parts by weight, molecular weight is average molecular weight, temperature is in degrees Celsius, and pressure is at or near atmospheric. Standard abbreviations may be used, e.g., bp: base pair(s); kb: kilobase(s); pl: picoliter(s); s or sec: second(s); min: minute(s); h or hr: hour(s); aa: amino acid(s); nt: nucleotide(s); and the like.

### Example 1—Rice OsACBP2 Mutants

Single mutant osacbp2 PFG_1D-05815 was purchased from Rice T-DNA Insertion Sequence Database (RISD DB; cbi.khu.ac.kr/RISD_DB.html), and osacbp2 mutants RMD_03Z11AZ19, RMD_03Z11LE18 and RMD_03Z11LG76 were purchased from the Rice Mutant Database (RMD; rmd.ncpgr.cn). For Western blot analysis, anti-OsACBP1 antibodies were synthesized by EzBiolab (www.ezbiolab.com/), and it has been shown to cross-react with OsACBP2 as OsACBP1 shares 79% amino acid sequence identity to OsACBP2. Rice seeds were tested as described in Lu, et al., Plant Cell 19:2484-2499, 2007 and Yi and An, J. Plant Biol. 56:85-90, 2013, in each of three repeats, and the results were consistent.

### Example 2—Growth of Rice Seedlings

Rice grains were incubated in 1% (v/v) nitric acid at room temperature in darkness for 1 d. After washing several times using distilled water, they were transferred to sterilized water and incubated in darkness at 37° C. for 2 days. Germinated seeds were moved to rice growth medium in a greenhouse maintained at 24-28° C.

### Example 3—Transmission Electron Microscopy

The ultrastructure of embryos from 1-d-old imbibed seeds of OsACBP2-OEs, osacbp2 mutants and wild-types was examined by TEM as described in Sieber, et al., Plant Cell 12:721-737, 2000 with some modifications. Samples were fixed using 2.5% (v/v) glutaraldehyde and 1.6% (v/v) paraformaldehyde in 0.1 M sodium cacodylate-HCl buffer (pH 7.4) overnight at 4° C., followed by post-fixation treatment with 1% (w/v) OsO₄ in cacodylate buffer for 2 h at room temperature. After gradient dehydration with ethanol, samples were infiltrated overnight with 1:1 (v/v) epoxy resin/propylene oxide mixture. Samples were subsequently embedded in epoxy resin and the resin polymerized overnight at 65° C. Ultrathin (60 nm) sections were prepared and stained with 2% (w/v) uranyl acetate and 2% (w/v) lead citrate, and subsequently examined using a Phillips CM100 transmission electron microscope.

### Example 4—Fatty Acid Analysis

Fatty acid (FA) analysis was conducted as described in Carvalho and Malcata, J. Agric. Food Chem. 53:5049-5059, 2005 with modifications. Thirty mg of lyophilized powder of rice seed embryos and endosperm was suspended respectively in a solution containing 1 ml of toluene, 2 ml of 1% (v/v) sulphuric acid in methanol together with 10 μl internal standard [C19:0 (1 mg ml-1 in hexane)]. The mixtures were incubated for 12 h at 50° C. for transmethylation of FAs. After several washes with 5% (w/v) NaCl and hexane, the hexane layer was washed with 4 ml of 2% (w/v) NaHCO₃ and then transferred into another test tube followed by vigorous vortexing. Nitrogen gas was blown into each tube to evaporate the hexane. Then 100 μl of hexane was re-added to the test tube to concentrate the FA residues. One μl of the hexane supernatant, containing the FAs, was analyzed by VT-WAXMS (Agilent Technologies) equipped with an HP-INNOWAX capillary column (HP19091N-133, 30 m×0.25 nm×0.25 μm). The samples were positioned and then automatically injected into the column. For sample detection, the oven temperature was increased from 100° C. to 230° C. with a rate of 4° C./min and post run at 235° C. for 4 min. The software GC/MSD On-line Data Analysis was used for data processing after data acquisition; the FAME library was used for compound identification.

### Example 5—OsACBP2 is Highly Expressed in Developing and Germinating Rice Seeds

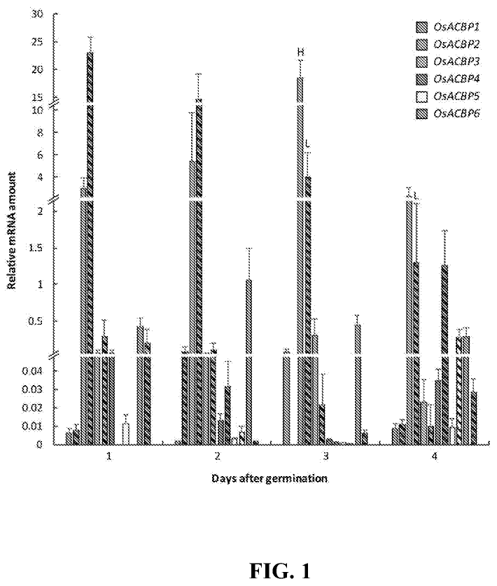

Six OsACBPs exist in rice (Meng, et al., New Phytologist 190:807-807, 2011) and they are distributed across Class I (OsACBP1, OsACBP2 and OsACBP3), Class II (OsACBP4), Class III (OsACBP5) and Class IV (OsACBP6) based on the presence of protein domain in their peptides. Using qRT-PCR, OsACBP2 was identified to be the only Class I OsACBP member that is highly expressed in both embryos and endosperm 1 to 4 days after germination (DAG) (FIG. 1). In contrast, OsACBP1, OsACBP3, OsACBP4, OsACBP5 and OsACBP6 showed relatively low expression (FIG. 1). In embryos, OsACBP2 peaked at 3 DAG, with a ˜4-fold increase over 1 DAG, while in the endosperm, it was highly expressed at 1 DAG and gradually declined (FIG. 1). TRIzol reagent (Invitrogen) was used for extraction of total RNA from 5 μg of homogenized sample. Subsequently, the total RNA was reverse-transcribed using the Superscript First-strand Synthesis System (Invitrogen) according to the manufacturer's protocol. Quantitative real-time PCR was conducted on a StepOne Plus Real-time PCR system using SYBR Green Mix (Applied Biosystems) and the program is as follows: 10 min at 95° C. followed by 40 cycles of 95° C. (15 s) and 56° C. (1 min). For each reaction, three experimental replicates were performed with gene-specific primers (Table 1), and Oryza sativa ACTIN (GenBank accession number X16280) was used as an internal control. The relative expression of the targeted genes was normalized using the ACTIN control. Provided in Table 1 below are primers used for qRT-PCR analysis, as well as other primers used in the present Examples:

TABLE 1

| Primer | Length | Sequence | Location | Orientation |
|---|---|---|---|---|
| ML1103 | 20-mer | 5′-TGTCAATACTGCTCGTCCTG-3′ | 177-196 in OsACBP1 mRNA (Locus ID | Forward |
| | | (SEQ ID NO: 10) | Os08g0162800) | |
| ML1104 | 20-mer | 5′-TAGTCGCTCATTGCTTCCTC-3′ | 262-281 in OsACBP1 mRNA (Locus ID | Reverse |
| | | (SEQ ID NO: 11) | Os08g0162800) | |
| ML1105 | 20-mer | 5′-ATGGGTTTGCAGGAGGAGTTT-3′ | 76-96 in OsACBP2 mRNA (Locus ID | Forward |
| | | (SEQ ID NO: 12) | Os06g0115300) | |
| ML1106 | 21-mer | 5′-CTGCTTGTAGAGGCCATAGAG-3′ | 160-180 in OsACBP2 mRNA (Locus ID | Reverse |
| | | (SEQ ID NO: 13) | Os06g0115300) | |
| ML1107 | 21-mer | 5′-TGGGTCTGCAGGAGGATTTTG-3′ | 2-22 in OsACBP3 mRNA (Locus ID | Forward |
| | | (SEQ ID NO: 14) | Os03g0576699) | |
| ML1108 | 21-mer | 5′-ACGGTGGCCTGCTTGTAGAGT-3′ | 93-113 in OsACBP3 mRNA (Locus ID | Reverse |
| | | (SEQ ID NO: 15) | Os03g0576699) | |
| ML1109 | 20-mer | 5′-GCATCTGGCTGCTGGTGTAG-3′ | 809-828 in OsACBP4 mRNA (Locus ID | Forward |
| | | (SEQ ID NO: 16) | Os04g0681900) | |
| ML1110 | 22-mer | 5′-GCATTTGCATTGACAAGAATCT-3′ | 904-925 in OsACBP4 mRNA (Locus ID | Reverse |
| | | (SEQ ID NO: 17) | Os04g0681900) | |
| ML1111 | 20-mer | 5′-GAGGCTATTCCAGGATGGAT-3′ | 1662-1681 in OsACBP5 mRNA (Locus ID | Forward |
| | | (SEQ ID NO: 18) | Os03g0243600) | |
| ML1112 | 22-mer | 5′-CTGTCATGTTGGTTGATTGTAT-3′ | 1759-1780 in OsACBP5 mRNA (Locus ID | Reverse |
| | | (SEQ ID NO: 19) | Os03g0243600) | |
| ML1113 | 20-mer | 5′-GGTGGTGGCAATAACAAAAG-3′ | 1438-1457 in OsACBP6 mRNA (Locus ID | Forward |
| | | (SEQ ID NO: 20) | Os03g0835600) | |
| ML1114 | 19-mer | 5′-GCAAGGGGAACACGACCTT-3′ | 1523-1541 in OsACBP6 mRNA (Locus ID | Reverse |
| | | (SEQ ID NO: 21) | Os03g0243600) | |
| ML1115 | 20-mer | 5′-AGGCCGTCCTCTCTCTGTAT-3′ | 522-541 in rice actin mRNA (Genbank | Forward |
| | | (SEQ ID NO: 22) | accession number AK100267) | |
| ML1116 | 20-mer | 5′-GGGGAGAGCATATCCTTCAT-3′ | 609-628 in rice actin mRNA (Genbank | Reverse |
| | | (SEQ ID NO:23) | accession number AK100267) | |
| ML2305 | 29-mer | 5′-TAGCTGCAGTAATGTCCTGATGC | -1728--1709 in 1.7 kb OsACBP2 5′- | Forward |
| | | GTTGCG-3′ (SEQ ID NO: 24) | flanking sequence; PstI site underlined | |
| ML2308 | 28-mer | 5′-TAGGGATCCCGATGGCGTCTCG | -24--6 in 1.7 kb OsACBP2 5′-flanking | Reverse |
| | | TTCTCG-3′ (SEQ ID NO: 25) | sequence; BamHI site underlined | |
| ML2523 | 27-mer | 5′-GTCATTTTGTCAATGTGATGAC | -961--935 in 1.7 kb OsACBP2 5′-flanking | Reverse |
| | | CAGGT-3′ (SEQ ID NO: 26) | sequence | |
| ML2524 | 27-mer | 5′-ACCTGGTCATCACATTGACAAAA | -961--935 in 1.7 kb OsACBP2 5′-flanking | Forward |
| | | TGAC-3′ (SEQ ID NO: 27) | sequence | |
| ML2525 | 29-mer | 5′-ACGAGATGTCATCCTGTACTGTC | -773--745 in 1.7 kb OsACBP2 5′-flanking | Forward |
| | | CATGTT-3′ (SEQ ID NO: 28) | sequence | |
| ML2526 | 29-mer | 5′-AACATGGACAGTACAGGATGACA | -773--745 in 1.7 kb OsACBP2 5′-flanking | Reverse |
| | | TCTCGT-3′ (SEQ ID NO: 29) | sequence | |
| ML2564 | 29-mer | 5′-TGTACATCAGCGCAAATGACATA | -1495--1467 in 1.7 kb OsACBP2 5′- | Reverse |
| | | AAAGGA-3′ (SEQ ID NO: 30) | flanking sequence | |
| ML2565 | 29-mer | 5′-TCCTTTTATGTCATTTGCGCTGAT | -1495--1467 in 1.7 kb OsACBP2 5′- | Forward |
| | | GTACA-3′ (SEQ ID NO: 31) | flanking sequence | |
| ML2566 | 30-mer | 5′-ATAGCCTCAGTTTGTTTGTCATCC | -843--814 in 1.7 kb OsACBP2 5′-flanking | Forward |
| | | CTATAT-3′ (SEQ ID NO: 32) | sequence | |
| ML2567 | 30-mer | 5′-ATATAGGGATGACAAACAAACTG | -843--814 in 1.7 kb OsACBP2 5′-flanking | Reverse |
| | | AGGCTAT-3′ (SEQ ID NO: 33) | sequence | |
| ML2591 | 29-mer | 5′-TAGCTGCAGAGTTCACTGTTCCTC | -866--847 in 1.7 kb OsACBP2 5′-flanking | Reverse |
| | | CCTCA-3′ (SEQ ID NO: 34) | sequence; PstI site underlined | |
| ML2835 | 29-mer | 5′-TAGCTGCAGATGATTTTCGGCCC | -809--790 in 1.7 kb OsACBP2 5′-flanking | Reverse |
| | | TTACCG-3′ (SEQ ID NO: 35) | sequence; PstI site underlined | |
| ML2850 | 29-mer | 5′-TAGCTGCAGATCGCCACACACG | -266--247 in 1.7 kb OsACBP2 5′-flanking | Reverse |
| | | AGAGGCT-3′ (SEQ ID NO: 36) | sequence; PstI site underlined | |
| M12854 | 29-mer | 5′-TAGCTGCAGGGCTCAAAAGATTC | -1217--1198 in 1.7 kb OsACBP2 5′- | Reverse |
| | | GTCTCG-3′ (SEQ ID NO: 37) | flanking sequence; PstI site underlined | |
| 355B | 25-mer | 5′-CAATCCCACTATCCTTCGCAAG | 281-285 in CaMV 35S promoter | Reverse |
| | | ACC-3′ (SEQ ID NO: 38) | | |

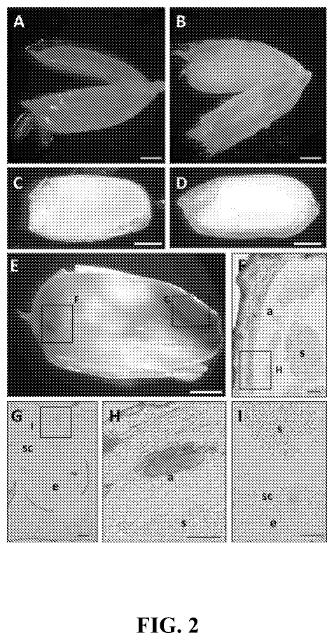

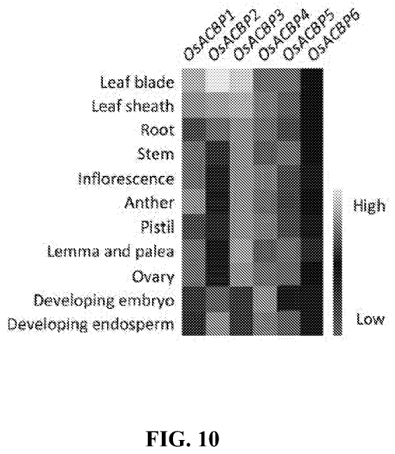

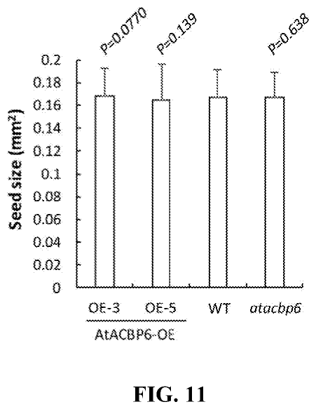

As OsACBP2 has been previously shown to be highly expressed in milk and soft dough seeds (Meng, et al., 2011, supra), the temporal and spatial expression of OsACBP2 was investigated by generating OsACBP2pro::GUS transgenic rice lines. In histochemical β-glucuronidase (GUS) assays, signals were not observed during early seed development (FIG. 2A, FIG. 2B and FIG. 2C) until 16 days after fertilization (DAF; FIG. 2D) and seed germination (FIG. 2E). In 1-d-old imbibed seed embryos and endosperm, OsACBP2pro::GUS was further detected in the scutellum and aleurone layer (FIG. 2F, FIG. 2G, FIG. 2H and FIG. 2I) but not in the starchy endosperm or embryonic apical region (FIG. 2). These expression profiles in developing seeds (FIG. 1 and FIG. 2) were consistent with the microarray data from the RiceXPro. The heat map further showed OsACBP2 was highly expressed in developing seeds and leaf blades (FIG. 10). In contrast, OsACBP1, OsACBP3, OsACBP5 and OsACBP6 displayed insignificant expression in developing seeds (FIG. 10). It is to be noted that AtACBP1 and AtACBP2, which are expressed in Arabidopsis seeds (Chye, et al., Plant J. 18:205-214, 1999; Chen, et al., New Phytol. 186:843-855, 2010), do not influence grain size and weight (FIG. 11).

The OsACBP2pro::GUS construct consisting of a 1.8-kb 5′-flanking region that was amplified by primers ML2305/ML2308 (Table 1) was generated by inserting the 1.8-kb OsACBP2pro fragment into a pGEM-T Easy vector (Promega), and eventually cloning into corresponding sites on binary vector DX2181 (Du, et al., Plant Physiol. 154:1304-1318, 2010) to yield plasmid pOS806. DNA sequence analysis was used to verify the PCR fragment cloned. This construct was sent to BioRun (www.biorun.net) for rice Zhonghua11 transformation. Histochemical GUS assays were carried out as described (Jefferson, et al., EMBO J. 6:3901-3907, 1987) with modifications. Plant samples were immersed and vacuum filtrated in the GUS staining solution [100 mM sodium phosphate buffer pH 7.0, 0.1% (v/v) Triton X-100, 2 mM potassium ferricyanide, 2 mM potassium ferrocyanide, 1 mg ml⁻¹ 5-bromo-4-chloro-3-indolyl-β-D-glucuronide] for 2 h, and incubated and observed over a period ranging from 3 to 16 h at 37° C. Subsequently, samples were cleared in 70% ethanol and photographed.

### Example 6—Five Skn-I-Like Motifs Regulate OsACBP2 Expression

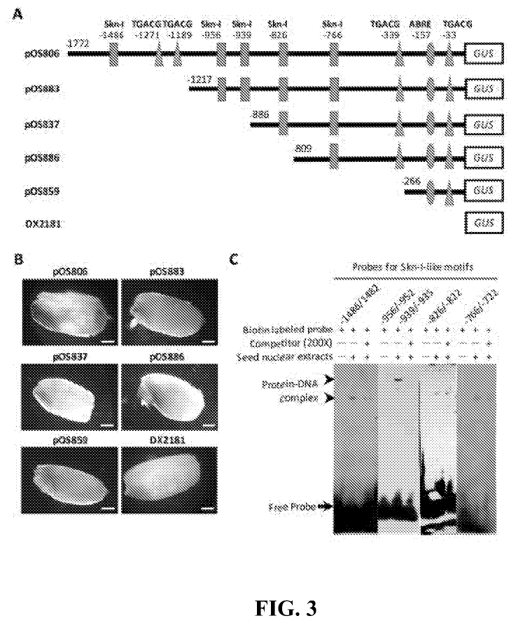

The OsACBP2 5′-flanking region was predicted using PlantCARE (Lescot, et al., Nucl. Acids Res. 30:325-327, 2002) to contain five putative copies of Skn-I-like motifs, determinants of seed-specific expression (Washida, et al., Plant Mol. Biol. 40:1-12, 1999), at nucleotide positions −1486/−1482, −956/−952, −939/−935, −826/−822 and −766/−762 (FIG. 3A). Additionally, there were four putative TGACG motifs at −1271/−1267, −1189/−1185, −339/−335 and −33/−29 (FIG. 3A), which may potentially recruit TGACG transcription factors in response to MeJA (Spoel, et al., Plant Cell 15:760-770, 2003). A potential abscisic acid-responsive (ABRE) element was also found at −157/−147 (FIG. 3A).

To evaluate the functions of these cis-elements, transgenic rice expressing GUS under the control of the OsACBP2pro and its truncated derivatives were generated (FIG. 3A). Using plasmids pOS806, pOS883, pOS837, pOS886, pOS859 and DX2181 (Du, et al., Plant Physiol. 154:1304-1318, 2010) transgenic rice lines, histochemical staining (Jefferson, et al., EMBO J. 6:3901-3907, 1987) of 1-d-old imbibed seeds indicated GUS expression in all lines except pOS859 transformants (FIG. 3B). Interestingly, only pOS806 displayed GUS expression in both endosperm and embryos, while GUS signals in pOS883, pOS837 and pOS886 were embryo-specific (FIG. 3B).

To investigate the function of the putative Skn-I-like motifs in the OsACBP2 5′-flanking region, electrophoretic mobility shift assays (EMSAs) were performed with LightShift® Chemiluminescent EMSA Kit (ThermoFisher Scientific) using double-strand biotin-labeled DNA probes containing five Skn-I-like motifs (−1484/−1480, −955/−951 and −938/−934, −827/−823 and −765/−761). Primers ML2564/ML2565 were used to generate probes containing Skn-I-like motif at −1486/−1482; ML2523/ML2524 for −956/−952 and −939/−935; ML2567/ML2568 for −826/−822; and ML2525/ML2526 for −766/−762 (Table 1). When crude nuclear extracts from 1-d-old imbibed seeds were tested, band shifts were observed with all four sets of probes, indicating DNA-protein interactions (FIG. 3C), while non-biotin-labeled probes outcompeted the protein binding (FIG. 3C), suggesting that all five Skn-I-like motifs could interact with nuclear proteins. Interestingly, genes that are regulated by Skn-I-like motifs, such as rice storage protein glutelin gene GluB-1 (Washida, et al., Plant Mol. Biol. 40:1-12, 1999), have not been previously reported to regulate grain size and weight.

### Example 7—OsACBP2 Overexpression Enhances Grain Size and/or Weight and Seedling Growth

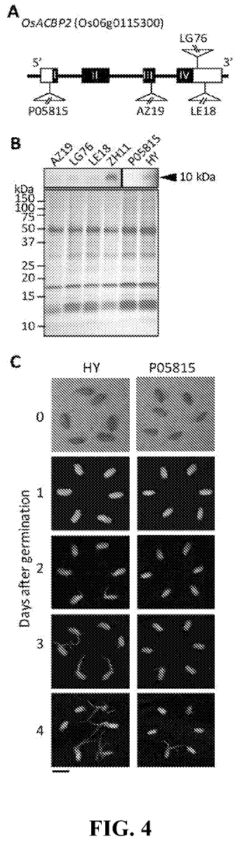

To study OsACBP2 function, an osacbp2 T-DNA insertional mutant PFG_1D-05815 (P05815) and three retrotransposon insertional mutants RMD_03Z11AZ19 (AZ19), RMD_03Z11LE18 (LE18) and RMD_03Z11LG76 (LG76) of OsACBP2 were characterized (FIG. 4A). The osacbp2 P05815 was derived from the Hwayoung wild-type (HY), and its T-DNA insertion was mapped to the 5′-untranslated region (UTR). The osacbp2 mutants, AZ19, LE18 and LG76 were originated from the Zhonghua11 wild-type (ZH11). The inserts were localized to the 3′-UTR for LE18 and LG76, while the insert for AZ19 was mapped to the third intron (FIG. 4A). Western blot analysis using 1-d-old rice seeds confirmed all four mutants knock-down OsACBP2 expression (FIG. 4B).

For detection of OsACBP2 following SDS-PAGE, plant total proteins were transferred to a Hybond™-ECL™ membrane (Amersham) using a Trans-Blot® cell (Bio-Rad) according to standard methods. After incubation in blocking buffer [5% (w/v) non-fat dry milk in TTBS buffer (20 mM Tris-HCl, pH 7.5, 500 mM NaCl, 0.05% (v/v) Tween-20)] at room temperature for 1 h, the membrane was washed 3×10 min with TTBS buffer and then incubated in anti-recombinant (His)₆-OsACBP1 antibodies [1:5000, synthesized by EzBiolab (www.ezbiolab.com/); sharing 79% amino acid sequence identity to OsACBP2] in blocking buffer with gentle shaking at room temperature for 2 h. The membrane was washed for 2×5 min with TTBS, then incubated in ECL™ anti-rabbit IgG Horseradish Peroxidase-linked whole antibody (from donkey) (1:3000, GE) in TTBS for 1 h with gentle agitation. After incubation with anti-rabbit antibodies, the membrane was washed for 3×10 min in TTBS. ECL™ Western Blotting Detection Reagents (GE Healthcare) was used for detection.

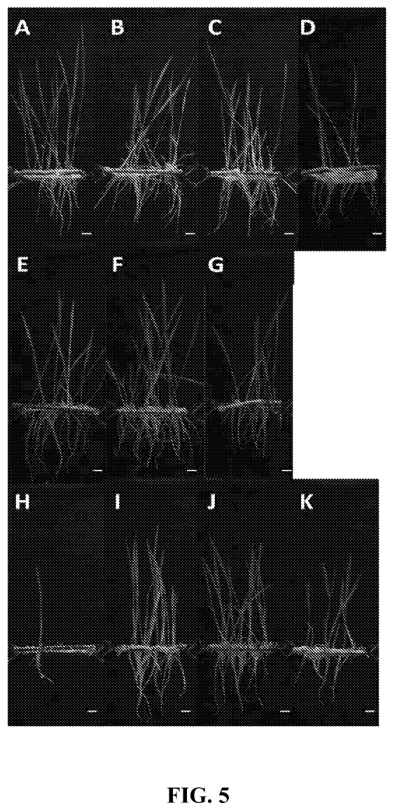

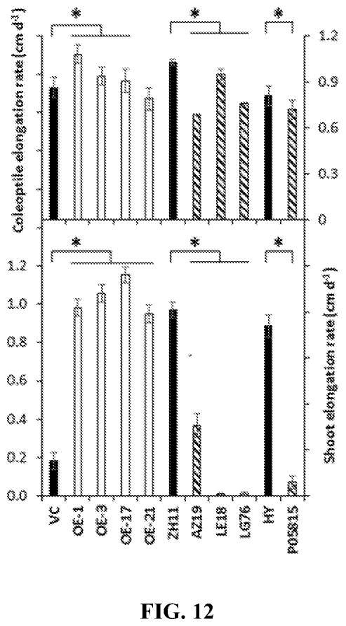

Seed germination and seedling growth were retarded in osacbp2 P05815 from 2 DAG (FIG. 4C). The P05815 seeds did not possess elongated coleoptiles as the wild-type until 4 DAG (FIG. 4C). For each group, six seeds in each of three repeats were tested, and their results were consistent. When 2-week-old wild-type controls (ZH11 and HY), the pCXSN vector control (VC), osacbp2 mutants (AZ19, LE18, LG76 and P05815) and OsACBP2-overexpressors (OsACBP2-OE; OE-1, OE-3, OE-17 and OE-21) were examined, OsACBP2-OEs showed increased coleoptile and shoot elongation rates over Zhonghua11 (ZH11) and the VC (FIG. 5). Mutants showed reduced rates for both shoot and coleoptile elongation in comparison to their corresponding wild-types (FIG. 12).

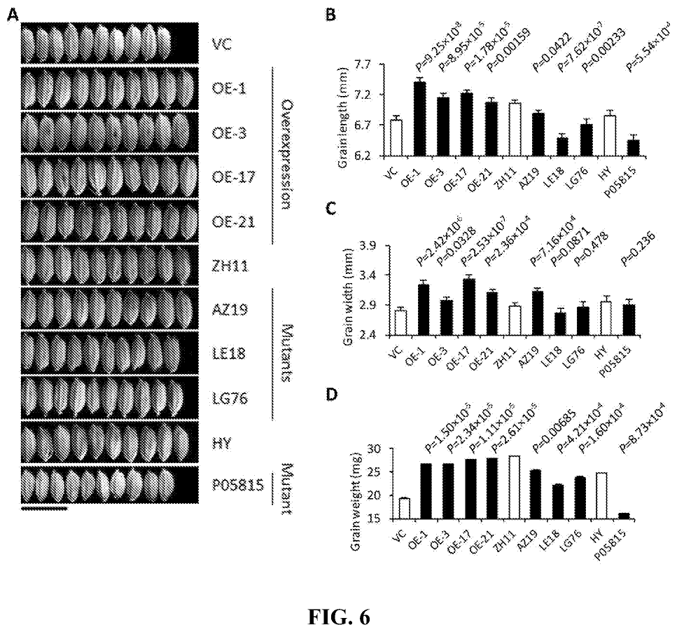

When grain characteristics were examined, the OsACBP2-OE transgenic rice displayed improved grain length (+10%), width (+10%), and weight (+10%), in comparison to the VC (FIG. 6). Correspondingly, the four osacbp2 lines (P05815, AZ19, LE18, and LG76) exhibited reduction in grain length (˜5%) and weight (˜10%), but not width, in comparison to their corresponding wild-types (FIG. 6). However, when the Class I Arabidopsis homologue was overexpressed in transgenic Arabidopsis, AtACBP6-OE lines and the atacbp6 mutant did not show significant differences from the wild-type in grain size (FIG. 11). It has been previously shown that no variations in seed morphology and size occurred among the double and triple mutants of atacbp4, atacbp5 and atacbp6 in comparison to the wild-type. As no ACBP has been reported to affect seed weight, OsACBP2 is the first ACBP demonstrated to regulate grain size and weight.

### Example 8—OsACBP2-OE Transgenic Rice Seeds Accumulate More Storage Lipids

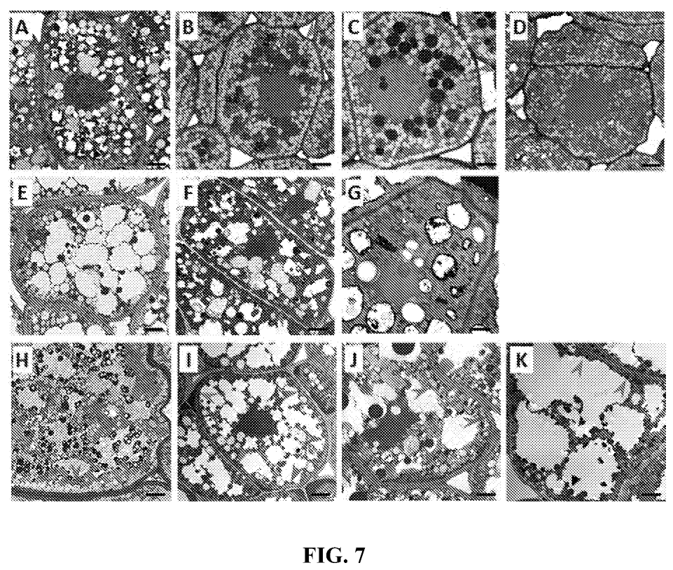

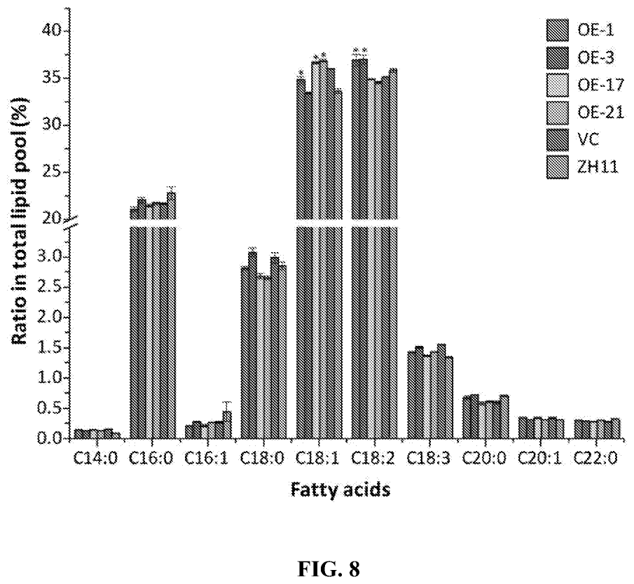

To investigate how OsACBP2 confers increase in grain size and weight, the scutellum cells from transgenic OsACBP2-OE rice seeds were examined using transmission electron microscopy (TEM), as described in Sieber, et al., Plant Cell 12:721-737, 2000. More oil bodies were observed in the OsACBP2-OEs than the VC, while fewer were detected in the osacbp2 mutants in comparison to the respective wild-types (FIG. 7). Using gas chromatography-mass spectrometry (GC-MS), the total fatty acid content in OsACBP2-OE seeds was found to be ˜10% higher than the VC, while osacbp2 P05815 and LE18 mutant seeds showed a 10%-20% decrease in total fatty acid content (Table 2). C18:0-FA accumulated in the OsACBP2-OEs over the VC (Table 2). When OsACBP2-OE embryos were further examined using GC-MS to test the level of the nine major FA species (C14:0-, C16:0-, C16:1-, C18:0-, C18:1-, C18:2-, C18:3-, C20:0- and C22:0-FA), the three most abundant species that comprise over 90% of the total FA pool, were C16:0-FA (20%-24%), C18:1-FA (32%-37%) and C18:2-FA (32%-37%) (FIG. 8). Amongst the four OsACBP2-OEs (OE-1, OE-3, OE-17 and OE-21), C18:1- and C18:2-fatty acids (FAs) accumulated in the embryos of 1-d-old imbibed seeds in comparison to Zhonghua11 (ZH11) wild-type and the pCXSN vector control (VC) (FIG. 8). The OE-1 and OE-3 embryos had accumulated ˜2.5% more C18:2-FA than the ZH11 and the pCXSN control (FIG. 8). Higher C18:1-FA was detected in OE-17 and OE-21 (FIG. 8). For other species of FAs (C14:0-, C16:0-, C16:1-, C18:0-, C18:3-, C20:0-, C22:0-FAs) measured, no significant difference was detected between the OsACBP2-OEs and the controls (FIG. 8). Overall, all four OsACBP2-OEs consistently showed an increased FA content (FIG. 8). The results revealed that the OsACBP2-OEs accumulated C18:1- and C18:2-FAs in mature seed embryos (FIG. 8).

TABLE 2

| | Total | | | | | | | |
|---|---|---|---|---|---|---|---|---|
| | content | Relative composition (mol %) | | | | | | |
| Genotype | (% DW) | C14:0 | C16:0 | C16:1 | C18:0 | C18:1 | C18:2 | C18:3 |
| OsACBP2 | 2.85 ± 0.03^{H} | 0.71 ± 0.03^{L} | 23.66 ± 0.94^{L} | 0.27 ± 0.02 | 0.90 ± 0.95 | 35.39 ± 1.61 | 36.64 ± 1.68 | 1.15 ± 0.03 |
| OE-1 | | | | | | | | |
| OsACBP2 | 2.92 ± 0.04^{H} | 0.65 ± 0.00^{L} | 24.13 ± 0.09^{L} | 0.23 ± 0.00 | 2.64 ± 0.04^{H} | 32.50 ± 0.16^{L} | 37.29 ± 0.07^{H} | 1.24 ± 0.02 |
| OE-3 | | | | | | | | |
| OsACBP2 | 2.80 ± 0.06^{H} | 1.05 ± 0.01^{H} | 24.57 ± 0.12 | 0.22 ± 0.01 | 2.66 ± 0.06^{H} | 34.34 ± 0.12 | 34.64 ± 0.11 | 1.17 ± 0.02 |
| OE-17 | | | | | | | | |
| OsACBP2 | 2.89 ± 0.06^{H} | 0.72 ± 0.02^{L} | 24.84 ± 0.30 | 0.23 ± 0.01 | 2.42 ± 0.07^{H} | 33.57 ± 0.78^{L} | 35.87 ± 0.34 | 1.16 ± 0.03 |
| OE-21 | | | | | | | | |
| VC-12 | 2.60 ± 0.06 | 0.77 ± 0.03 | 25.06 ± 0.38 | 0.22 ± 0.05 | 0.66 ± 0.77 | 35.26 ± 0.80 | 35.46 ± 0.72 | 1.29 ± 0.06 |
| WT ZH11 | 2.67 ± 0.11 | 0.85 ± 0.01 | 24.35 ± 0.37 | 0.22 ± 0.00 | 2.94 ± 0.23 | 32.76 ± 0.38 | 36.41 ± 0.36 | 1.22 ± 0.02 |
| WT HY | 2.76 ± 0.11 | 0.74 ± 0.02 | 24.90 ± 0.37 | 0.14 ± 0.10 | 2.43 ± 0.08 | 30.45 ± 0.72 | 38.54 ± 0.22 | 1.54 ± 0.04 |
| osacbp2-1 | 1.95 ± 0.01^{L} | 0.83 ± 0.01 | 26.13 ± 0.13^{H} | 0.22 ± 0.01 | 2.13 ± 0.00 | 30.98 ± 0.16^{L} | 36.93 ± 0.13 | 1.55 ± 0.01^{H} |
| osacbp2-2 | 3.08 ± 0.04^{H} | 0.62 ± 0.00^{L} | 23.94 ± 0.20 | 0.31 ± 0.00^{H} | 2.28 ± 0.13 | 35.03 ± 0.12^{H} | 35.05 ± 0.07 | 1.42 ± 0.29 |
| osacbp2-3 | 2.58 ± 0.10^{L} | 1.45 ± 0.05^{H} | 25.58 ± 0.55 | 0.31 ± 0.02^{H} | 1.16 ± 1.39^{L} | 31.11 ± 1.11 | 37.31 ± 0.51 | 1.72 ± 0.07 |

| | Relative composition (mol %) | |
|---|---|---|
| Genotype | C20:0 | C20:1 |
| OsACBP2 | 0.62 ± 0.03^{H} | 0.41 ± 0.05 |
| OE-1 | | |
| OsACBP2 | 0.56 ± 0.01 | 0.53 ± 0.08 |
| OE-3 | | |
| OsACBP2 | 0.63 ± 0.01^{H} | 0.42 ± 0.01 |
| OE-17 | | |
| OsACBP2 | 0.49 ± 0.01^{L} | 0.45 ± 0.01 |
| OE-21 | | |
| VC-12 | 0.55 ± 0.02 | 0.47 ± 0.00 |
| WT ZH11 | 0.59 ± 0.03 | 0.40 ± 0.02 |
| WT HY | 0.55 ± 0.02 | 0.45 ± 0.01 |
| osacbp2-1 | 0.48 ± 0.01^{L} | 0.48 ± 0.05 |
| osacbp2-2 | 0.57 ± 0.01 | 0.49 ± 0.00^{H} |
| osacbp2-3 | 0.63 ± 0.03^{H} | 0.41 ± 0.02 |

### Example 9—OsACBP2-OE Transgenic Rice Embryos Accumulate Embryo Globulins

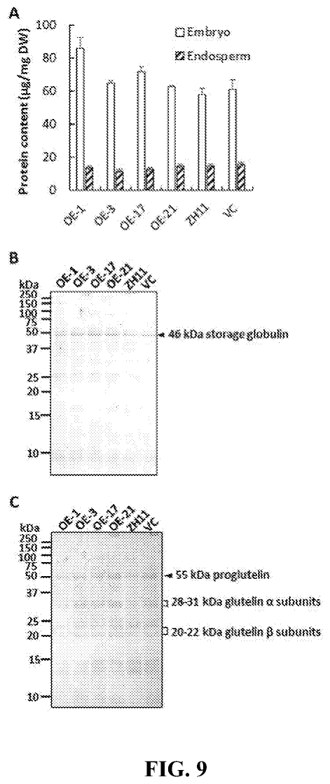

As protein bodies accumulated in OsACBP2-OE mature seed scutellum cells (FIG. 7), the total protein concentration on the dry weight (DW) basis in OsACBP2-OE embryos and endosperm was estimated using BCA Protein Assay Kit (Pierce). None of the four OsACBP2-OEs (OE-1, OE-3, OE-17 and OE-21) displayed significant changes in total protein content, of the embryo or endosperm from wild-type ZH11 and the VC (Student's t test, P>0.01; FIG. 9A). The protein content in OsACBP2-OE embryos was ˜60-80 μg/mg dry weight (DW), similar to ZH11 and the VC (FIG. 9A). The endosperm of imbibed seeds showed total protein of 12-15 μg/mg dry weight (FIG. 9A). However, when the same amount of protein from each sample was examined using SDS-PAGE, 46-kDa bands likely representing rice storage globulin (Sun, et al., Plant Cell Physiol. 37:612-620, 1996) appeared more prominently in OsACBP2-OE embryos (FIG. 9B). However, no obvious difference in the 11S glutelin bands (20-22 kDa, 28-31 kDa and 55 kDa) (Takaiwa, et al., in Seed Proteins, pp. 401-425, 1999) were noted (FIG. 9C).

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method of increasing grain size, weight, or lipid content in grain bran or whole seed, in a plant, comprising upregulating expression of an acyl-CoA-binding protein 2 gene in the plant, wherein the grain size, weight, or lipid content in grain bran or whole seed of
the plant is increased when compared to a control plant that lacks the upregulating.

2. The method of claim 1, wherein the upregulating comprises introducing into the plant a recombinant nucleic acid construct comprising the acyl-CoA-binding protein 2 gene operably linked to a heterologous promoter.

3. The method of claim 1, wherein the acyl-CoA-binding protein 2 gene is an Oryza sativa acyl-CoA-binding protein 2 gene.

4. The method of claim 3, wherein the acyl-CoA-binding protein 2 gene comprises a polynucleotide sequence having at least 77% sequence identity to SEQ ID NO:1 or the acyl-CoA-binding protein 2 gene comprises the polynucleotide sequence of SEQ ID NO:1.

5-9. (canceled)

10. The method of claim 3, wherein the acyl-CoA-binding protein 2 gene encodes a polypeptide having at least 82% sequence identity to SEQ ID NO:2 or the acyl-CoA-binding protein 2 gene encodes the polypeptide sequence of SEQ ID NO:2.

11-14. (canceled)

15. The method of claim 2, wherein the heterologous promoter is a constitutive, tissue-specific or inducible promoter.

16. The method of claim 1, wherein the grain size of the plant is increased or wherein the grain size of the plant is increased by about 20% compared to grain from a control plant that lacks the upregulating.

17. (canceled)

18. The method of claim 1, wherein the grain weight of the plant is increased by about 10% compared to grain from a control plant that lacks the upregulating, and/or wherein the lipid content in grain bran of the plant is increased by about 10% compared to grain bran from a control plant that lacks the upregulating, and/or wherein the lipid content in whole seed of the plant is increased by about 10% compared to whole seed from a control plant that lacks the upregulating.

19-23. (canceled)

24. The method of claim 1, wherein the plant is a monocotyledonous plant, is in the Poaceae family, is a rice plant, or is an Oryza sativa plant.

25-27. (canceled)

28. The method of claim 1, wherein the plant is a dicotyledonous plant.

29. A plant, or part thereof, produced by the method of claim 4.

30. A plant comprising a recombinant nucleic acid construct comprising:
(a) a polynucleotide sequence having at least 77% sequence identity to SEQ ID NO:1; or
(b) a polynucleotide sequence encoding a polypeptide having at least 82% sequence identity to SEQ ID NO:2;
wherein the polynucleotide sequence is operably linked to a heterologous promoter; and
wherein the grain size, weight, or lipid content in grain bran or whole seed, of the plant is increased when compared to a plant that lacks the recombinant nucleic acid construct.

31. A seed that produces the plant of claim 30.

32. (canceled)

33. A plant part of, or seed produced by, the plant of claim 30, wherein the plant part or seed comprises the recombinant nucleic acid construct.

34. A progeny plant of the plant of claim 30, wherein the progeny plant comprises the recombinant nucleic acid construct, and wherein the progeny plant has increased grain size, weight, or lipid content in grain bran or whole seed, when compared to a plant that lacks the recombinant nucleic acid construct.

35. A method of producing grain having increased size, weight, or lipid content in grain bran or whole seed, the method comprising:
(a) obtaining a plant comprising upregulated expression of an acyl-CoA-binding protein 2 gene in the plant, wherein the size or weight of the grain, or the lipid content in grain bran or whole seed is increased when compared to a plant that lacks the upregulated expression;
(b) growing the plant under plant growth conditions to produce grain from the plant; and
(c) collecting grain from the plant.

36. (canceled)

37. The method of claim 35, wherein the plant is a rice plant or is an Oryza sativa plant.

38. (canceled)

39. A method of producing a plant having increased grain size, weight, or lipid content in grain bran or whole seed, comprising crossing the plant of claim 30 with a second plant to produce at least a first progeny plant comprising increased grain size, weight, or lipid content in grain bran or whole seed, when compared to a control plant that lacks the recombinant nucleic acid construct.

40. The method of claim 39, wherein the plant is a rice plant.

41. The method of claim 1, wherein the plant is a rice plant, wherein the upregulating expression of an acyl-CoA-binding protein 2 gene is accomplished by introducing into the rice plant a recombinant nucleic acid construct comprising:
(a) a polynucleotide sequence having at least 77% sequence identity to SEQ ID NO:1; or
(b) a polynucleotide sequence encoding a polypeptide having at least 82% sequence identity to SEQ ID NO:2;
wherein the polynucleotide sequence is operably linked to a heterologous promoter.
